(19) United States
(12) Patent Application Publication
(10) Pub. No.: US 2017/0189376 A1
(43) Pub. Date: Jul. 6, 2017

(54) BUTYROYLOXYMETHYL DIETHYL PHOSPHATE COMPOUNDS AND USES THEREOF

(71) Applicants: MOR RESEARCH APPLICATIONS LTD., Tel Aviv (IL); BAR-ILAN UNIVERSITY, RAMAT GAN (IL)

(72) Inventors: Lilach Moyal ELCHARAR, Herzelia (IL); Emmilia HODAK, Tel-Aviv (IL); Ada REPHAELI, Herzelia (IL); Abraham NUDELMAN, Rehovot (IL)

(21) Appl. No.: 15/397,780

(22) Filed: Jan. 4, 2017

Related U.S. Application Data
(60) Provisional application No. 62/274,395, filed on Jan. 4, 2016.

(51) Int. Cl. A61K 31/385; A61K 31/704; A61K 45/06

(52) U.S. Cl. CPC A61K 31/385; A61K 45/06; A61K 31/704

(57) ABSTRACT

Methods for treating ameliorating, reducing and/or preventing a cutaneous T-cell lymphoma in a subject in need thereof, comprising administration butyroyloxymethyl diethyl phosphate (AN-7) alone or combined with an additional anti-cancer therapy.

### CROSS-REFERENCE TO RELATED APPLICATIONS

This application claims the benefit of U.S. Provisional Patent Application No. 62/274,395 filed Jan. 4, 2016, the contents of which are incorporated herein by reference in their entirety.

### FIELD OF INVENTION

The present invention is directed to, inter alia, compositions and methods for treating cutaneous T-cell lymphoma (CTCL).

### BACKGROUND OF THE INVENTION

Mycosis fungoides (MF), the most common type of cutaneous T-cell lymphoma (CTCL), is manifested clinically by patches that may gradually develop into plaques and eventually tumors. Sézary syndrome (SS) is a rare aggressive leukemic-phase type of MF. There is no known cure for MF/SS. Skin-directed therapy is the key to management of early-stage MF, and systemic therapy is essential in advanced MF and in cases of SS. Although there are several systemic therapeutic options primarily for advanced MF and SS for slowing disease progression and preserving quality of life as long as possible, response rates are relatively low. Therefore, novel effective treatments tailored for advanced-stage MF and SS and recurrent/refractory early-stage MF are required.

Histone deacetylase inhibitors (HDACIs) have been found to induce cell death in both solid and hematological malignancies, either extrinsically (death receptor pathway) or intrinsically (caspase activation, mitochondrial pathway), via transcription-dependent and transcription-independent mechanisms. Suberoylanilide hydroxamic acid, (SAHA, vorinostat), approved by the US Food and Drug Administration (FDA) in 2006 for the treatment of CTCL, is an orally bioavailable HDACI of classes I, II, and V. It induces accumulation of acetylated histones, cell-cycle arrest, and apoptosis selectively in cancer cell lines. Depsipeptide (Romidepsin) was the second HDACI approved by the FDA in 2009 for CTCL. These HDACIs, given as a single agent, yield overall response rate of 30-35%, but a complete response rate of only 2-6%. Given the limited clinical efficacy of these two HDACIs and their high rates of adverse effects, there is an ongoing effort to develop new HDACIs with improved efficacy and selectivity. Combination therapy may yield benefits from potentiating the efficacy of HDACI with other agents. However, currently data regarding combination treatments is strikingly sparse.

Prompted by findings that HDACIs sensitize tumor lines to DNA-damage inducers, it has been suggested that combining HDACIs with anti-tumor agents such as doxorubicin (Dox), a widely used anthracycline derivative, may yield better clinical results. Dox acts via formaldehyde-mediated alkylation of DNA with consequent adduct formation, and have shown high effectiveness against a broad range of cancers. Clinical studies with the HDACI-Dox combination treatment have reported promising results in various types of cancer, but data specifically for CTCL remain sparse.

Butyroyloxymethyl diethyl phosphate (AN-7) is a novel HDACI, which is a water-soluble, orally active prodrug of the HDACI butyric acid. Upon hydrolytic degradation, it releases butyric acid, formaldehyde, and phosphoric acid. Like other derivatives of butyric acid, AN-7 inhibits HDAC classes I and II and was found to exert anticancer activities in vitro and in vivo, in mouse model.

U.S. Pat. No. 08/814,386 provides compounds and compositions comprising AN-7 for treating cancer. Furthermore, it was previously shown that AN-7 is a selective anti-cancer drug displaying preferential cytotoxicity against leukemic and glioblastoma cells compared to their normal cellular counterparts-normal mononuclear and astrocytes cells. Additionally, AN-7 was shown to exhibit selective toxic and apoptotic effect in murine mammary 4T1, and human glioblastoma U251 cancer cell lines, as compared to neonatal rat cardiomyocytes, cardiofibroblasts and astrocytes. Moreover, it interacts synergistically with Dox in mice bearing mammary tumors and in the MCF-7 cell line.

### SUMMARY OF THE INVENTION

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

According to a first aspect, there is provided a method for treating ameliorating, reducing and/or preventing a cutaneous T-cell lymphoma in a subject in need thereof, said method comprising the step of: administering to the subject a therapeutically effective amount of a butyroyloxymethyl diethyl phosphate (AN-7) a derivative or salt thereof, thereby treating, ameliorating, reducing and/or preventing a cutaneous T-cell lymphoma in a subject in need thereof.

In some embodiments, the cutaneous T-cell lymphoma (CTCL) is selected from the group consisting of: Mycosis fungoides and Sézary syndrome.

In some embodiments, the method further comprises the step of simultaneously, sequentially or separately administering or applying an additional anti-cancer therapy selected from the group consisting of: a radiation therapy and an anti-cancer agent.

In some embodiments, the anti-cancer agent is a topoisomerase inhibitor. In some embodiments, the topoisomerase inhibitor is selected from the group consisting of: doxorubicin, epirubicin, daunomycin, amscrine, and mitoxantrone.

According to some aspects, there is provided a method for treating ameliorating, reducing and/or preventing cutaneous T-cell lymphoma in a subject in need thereof, said method comprising the step of: simultaneously, sequentially or separately administering to the subject a therapeutically effective amount of butyroyloxymethyl diethyl phosphate (AN-7) and an additional anti-cancer therapy, thereby treating, ameliorating, reducing and/or preventing cutaneous T-cell lymphoma in a subject in need thereof.

In some embodiments, the method is for increasing a therapeutic response to said anti-cancer therapy. In some embodiments, the method is for increasing therapeutic potency, efficacy, or selectivity of said anti-cancer therapy.

In some embodiments, the cutaneous T-cell lymphoma (CTCL) is selected from the group consisting of: Mycosis fungoides and Sézary syndrome.

In some embodiments, the anti-cancer agent is a topoisomerase inhibitor. In some embodiments, the topoisomerase inhibitor is selected from the group consisting of: doxorubicin, epirubicin, daunomycin, amscrine, and mitoxantrone.

According to some aspects, there is provided a method for inducing cell death, proliferation arrest, or growth arrest in a CTCL cell, the method comprising the step of contacting the CTCL cell with AN-7 a derivative or salt thereof, thereby inducing cell death, proliferation arrest, or growth arrest.

In some embodiments, the method further comprises the step of simultaneously, sequentially, or separately contacting the CTCL cell with an anti-cancer agent.

In some embodiments, the cutaneous T-cell lymphoma (CTCL) is selected from the group consisting of: Mycosis fungoides and Sézary syndrome.

In some embodiments, the anti-cancer agent is a topoisomerase inhibitor. In some embodiments, the topoisomerase inhibitor is selected from the group consisting of: doxorubicin, epirubicin, daunomycin, amscrine, and mitoxantrone.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D are graphs showing viability curves based on the MTT assay of MyLa cells, Hut78 cells, (A, B), and SPBL (n=3) (C, D) compared to NPBL (n=8) following treatment with SAHA (A, C) and AN-7 (B, D) for 72 hours;

FIGS. 2A-H demonstrate the toxic and apoptotic effect of SAHA and AN-7 on MF/SS cell lines as a function of exposure time: FIGS. 2A-D are graphs showing Viability curves based on trypan blue staining of MyLa and Hut78 cells following short or long exposure to SAHA (A, C) or AN-7 (B, D); FIGS. 2E-H are graphs showing Percent of apoptotic MyLa cells (early+late apoptosis) based on FACS analysis of annexin V and PI staining after short or long exposure to SAHA (E) or AN-7 (F), and apoptotic Hut78 cells after short or continuous exposure to SAHA (G) or AN-7 (H);

FIGS. 3A-F are FACS plots shown with percent of cells in each quadruplet following treatment of PBLs obtained from two SS patients (PBL-1 and PBL-2) untreated (A, D), treated with SAHA (B, E), or with AN-7 (C, F);

FIGS. 3G-H are bar graphs showing the percent of cells in apoptotic cells (early+late apoptosis) of FIGS. 3A-C (G) and FIGS. 3D-F (H);

FIG. 4A-D show immunoblot of apoptotic and pro-apoptotic proteins in MyLa and Hut78 cells treated with SAHA 10 μM or with AN-7 300 μM for the indicated periods (A), basal HDAC1 protein expression in NPBL and MF/SS cell lines (B) in MF/SS cell lines treated with SAHA 10 μM or AN-7 300 μM (C), and Acetylated H3 in the nuclear lysate of MF/SS cell lines treated with and the same concentrations of SAHA and AN-7 for the indicated periods (D).

FIGS. 5A-F are graphs showing viability curves based on the MTT assay of MyLa cells, Hut78 cells, and SPBL treated for 72 h with drug combinations, in comparison to NPBL as follows: MyLa cells treated with Dox+AN-7, 1:3000 (molar ratio) (A) and with Dox+SAHA, 1:150 (B); Hut78 cells treated with Dox+AN-7, 1:2600 (C) and Dox+SAHA, 1:38 (D); SPBL treated with Dox+AN-7, 1:1781 (E) and Dox+SAHA, 1:20 (F). NPBL were treated at same molar ratio as SPBL (A-F);

FIGS. 5G-J are CI-viability fraction plots of combined treatment based on viability curves of FIGS. 5A-F in MyLa cells (G), Hut78 cells (H), SPBL (I), and NPBL (J);

FIG. 6A shows representative images of MyLa and Hut78 cells treated with either AN-7, Doxorubicin (Dox), or AN-7 and Dox for 0, 24, 48, 72 or 96 hours and visualized under a fluorescence microscope;

FIGS. 6B-C are bar graphs representing the length and intensity of SYBR green-stained DNA tails relative to heads which is shown as the relative comet tail moment (n=100), the mean tail moment of untreated cells was reduced from each tail moment at indicated time point for MyLa cells (B) and Hut78 cells(C);

FIG. 7A-B are immunoblots of p-KAP1, γH2AX and Actin in a lysate of MyLa cells (A) and Hut78 (B) that were treated with AN-7, doxorubicin (Dox), or a combination of AN-7 and Dox, and incubated for the indicated time points following the treatment;

FIG. 8A shows representative images of γ-H2AX immunostaining in MyLa and Hut78 cells treated with AN-7, Dox or a combination of AN-7 and Dox, incubated for the indicated time points following the treatment;

FIG. 8B-C are bar graphs showing the percentage of cells having induction of at least 5 γ-H2AX in a lysate of MyLa (B) and Hut78 (C) cells that were treated with AN-7, Dox or a combination of AN-7 and Dox, and incubated for the indicated time points following the treatment;

FIGS. 9A-B are immunoblots of NBS1, Rad51, Mre11, DNA-PK, Ku70 and actin in a whole cell lysate of MyLa cells (A) and Hut78 (B) that were treated with AN-7, Dox or a combination of AN-7 and Dox, and incubated for the indicated time points following the treatment; and

FIG. 10 is a bar graph showing flow cytometry quantification of DSBs' repair induced by I-SceI in U2OS GFP-reporter cells that were transfected with I-SceI plasmid untreated or treated with 1 mM AN-7.

### DETAILED DESCRIPTION OF THE INVENTION

The present invention, in some embodiments thereof, relates to butyroyloxymethyl diethyl phosphate (AN-7) (CAS#: 213262-83-0) compound, derivatives or salts thereof, and compositions comprising the same, for treating ameliorating or preventing a T-cell lymphoma (e.g., cutaneous T-cell lymphoma) in a subject in need thereof.

The present invention, in some embodiments thereof, relates to the AN-7 compound, derivatives or salts thereof, and compositions comprising the same, for increasing effectiveness of an anti-cancer therapy in a subject in need thereof. The present invention further provides a combined treatment comprising AN-7 and at least one additional anti-cancer therapy. The anti-cancer therapy and the AN-7 compound may be applied or administered simultaneously, sequentially, or separately.

As used herein, the terms “combination treatment”, “combination therapy”, “combined treatment”, “combined preparation” or “combinatorial treatment”, used interchangeably, refer to a treatment of an individual with at least two different therapeutic agents. According to one aspect of the invention, the individual is treated with a first therapeutic agent, e.g., AN-7 as described herein. The combination treatments may be carried out simultaneously, sequentially, or separately.

In one embodiment, the AN-7 compound described herein is provided to the subject per se. In one embodiment, the compound described herein is provided to the subject as part of a pharmaceutical composition where it is mixed with a pharmaceutically acceptable carrier. In some embodiments, the AN-7 compound described herein is co-administered with an additional anti-cancer therapy and/or an additional active agent.

The present invention is based, in part, on the surprising finding that AN-7 has a toxic and apoptotic effect on neoplastic T-cells in cutaneous T-cell lymphoma (CTCL) cell lines. Surprisingly, apoptosis and viability assays demonstrated that AN-7 has a faster kinetic than other HDAC inhibitors (e.g., SAHA) and is highly effective and selective after both short and continuous treatment. The present invention is also based in part on the surprising finding that AN-7 acts synergistically and selectively with a topoisomerase II inhibitor (i.e., doxorubicin) in vitro and ex vivo to kill neoplastic T-cells of CTCL.

As exemplified in the example section below, AN-7 acted synergistically with doxorubicin (Dox) in peripheral blood lymphocytes obtained from patients with Sezary syndrome (SPBL) and in a cell line of cutaneous T-cell lymphomas of Mycosis Fungoides and Sézary syndrome (MF/SS).

Further, AN-7 acted antagonistically with doxorubicin in peripheral blood lymphocytes taken from healthy individuals (NPBL). As further exemplified in the example section below, and without limiting the invention to any mechanism of action, AN-7 inhibit the repair machinery of double-strand breaks (DSBs) induced by Dox treatment, leading to accumulation of unrepaired damage in CTCL cell lines.

### Pharmaceutical Compositions

According to some aspect, the invention provides a pharmaceutical composition comprising as an active ingredient a therapeutically effective amount of AN-7 or a derivative or salt thereof, and a pharmaceutically acceptable carrier and/or diluents, for treating ameliorating or preventing a T-cell lymphoma (e.g., cutaneous T-cell lymphoma). In some embodiments, the pharmaceutical composition facilitates administration of a compound to an organism.

In some embodiments, the composition is being packaged in a packaging material and identified in print, in or on the packaging material, for use in the treatment of a medical condition associated with any disease, medical condition, or disorder as described hereinthroughout. In one embodiment, the composition is being packaged in a packaging material and identified in print, in or on the packaging material, for the treatment of CTCL, including but not limited to, Mycosis fungoides or Sézary syndrome.

The AN-7 or a derivative or salt thereof described herein (also termed herein “AN-7 compounds”) may be administered or otherwise utilized either as is, or as a pharmaceutically acceptable salt, enantiomer, diastereomer, solvate, hydrate or a prodrug thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. The neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of the present invention. The phrase “pharmaceutically acceptable salts” is meant to encompass salts of the active compounds which are prepared with relatively nontoxic acids or bases, depending on the particular substituents found on the compounds described herein.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compound as described herein to be converted into either base or acid addition salts.

In some embodiments, the neutral forms of the compounds described herein are regenerated by contacting the salt with a base or acid and isolating the parent compounds in a conventional manner. The parent form of the compounds differs from the various salt forms in certain physical properties, such as solubility in polar solvents, but otherwise the salts are equivalent to the parent form of the conjugate for the purposes of the present invention.

The term “prodrug” refers to an agent, which is converted into the active compound (the active parent drug) in vivo. Prodrugs are typically useful for facilitating the administration of the parent drug. The prodrug may also have improved solubility as compared with the parent drug in pharmaceutical compositions. Prodrugs are also often used to achieve a sustained release of the active compound in vivo.

In some embodiments, the compounds described herein possess asymmetric carbon atoms (optical centers) or double bonds; the racemates, diastereomers, geometric isomers and individual isomers are encompassed within the scope of the present invention.

As used herein and in the art, the term “enantiomer” describes a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

In some embodiments, the compounds described herein can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

According to another aspect, there is provided a pharmaceutical composition comprising, as an active ingredient, any of the compounds described herein and a pharmaceutically acceptable carrier.

Accordingly, in methods and uses described herein, one or more of the compounds described herein can be provided to an individual either per se, or as part of a pharmaceutical composition where it is mixed with a pharmaceutically acceptable carrier.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

As used herein, the term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the compound/composition of the invention, preferably in a substantially purified form, together with a suitable amount of carrier so as to provide the form for proper administration to the subject.

According to an embodiment of the invention, pharmaceutical compositions contain 0.1% -95% of the compound/composition of the present invention, derivatives, or analogs thereof. According to another embodiment of the invention, pharmaceutical compositions contain 1%-70% of the compound/composition. According to another embodiment of the invention, the composition or formulation to be administered may contain a quantity of compound/composition according to embodiments of the invention in an amount effective to treat the condition or disease of the subject being treated.

An embodiment of the invention relates to AN-7 a derivative, or salt thereof, presented in unit dosage form and prepared by any of the methods well known in the art of pharmacy. In an embodiment of the invention, the unit dosage form is in the form of a tablet, capsule (e.g., soft gel capsule), lozenge, wafer, patch, ampoule, vial or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

According to one embodiment, the compositions described herein are administered in the form of a pharmaceutical composition comprising at least one of the AN-7 compounds described herein together with a pharmaceutically acceptable carrier or diluent. In another embodiment, the compositions of this invention can be administered either individually or together (e.g., AN-7 and an additional anti-cancer therapy) in any conventional oral, parenteral or transdermal dosage form for any duration of time.

As used herein, the terms “administering,” “administration,” and like terms refer to any method which, in sound medical practice, delivers a composition containing an active agent to a subject in such a manner as to provide a therapeutic effect.

Depending on the location of the tissue of interest, the compounds/composition of the present invention can be administered in any manner suitable for the provision of the compounds/composition to cells within the tissue of interest. Thus, for example, a composition containing the composition/compound of the present invention can be introduced, for example, into the systemic circulation, which will distribute the composition/compound to the tissue of interest. Alternatively, a composition can be applied topically to the tissue of interest (e.g., injected, or pumped as a continuous infusion, or as a bolus within a tissue, applied to all or a portion of the surface of the skin, etc.).

In some embodiments, the pharmaceutical compositions comprising AN-7 as the active agent are administered via oral, rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of compositions/compounds administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the AN-7 compound is administered intravenously. In some embodiments, the AN-7 compound is administered orally. In some embodiments, the AN-7 compound is in the form of soft gel capsules.

For topical application, a compound/composition of the present invention, derivative, analog or a fragment thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

According to some embodiments, the compounds described herein, derivatives, salts or analogs thereof can be delivered in a controlled release system. In another embodiment, an infusion pump can be used to administer the compound/composition such as the one that is used, for example, for delivering insulin or chemotherapy to specific organs or tumors. In another embodiment, the compounds/compositions described herein are administered in combination with a biodegradable, biocompatible polymeric implant, which releases the compound/composition over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

The presently described compounds/compositions, derivatives, salts or analogs thereof may also be contained in artificially created structures such as liposomes, ISCOMS, slow-releasing particles, and other vehicles which increase the half-life of the compounds/compositions in serum. Liposomes include emulsions, foams, micelles, insoluble monolayers, liquid crystals, phospholipid dispersions, lamellar layers and the like. Liposomes for use with the presently described compositions/compounds are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al,

Current Protocols in Protein Science, 1999, John Wiley & Sons, Inc., New York, and see also U.S. Pat. Nos. 4,235,871, 4,501,728, 4,837,028, and 5,019,369.

The compositions also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

In one embodiment, the present invention provides combined preparations. In some embodiments, the pharmaceutical composition comprises at least one AN-7 compound and a topoisomerase inhibitor as a combined preparation for simultaneous, sequential or separate use in cancer therapy. In some embodiments, the pharmaceutical composition comprises at least one AN-7 compound and doxorubicin as a combined preparation for simultaneous, sequential or separate use in cancer therapy.

In one embodiment, “a combined preparation” defines especially a “kit of parts” in the sense that the combination partners as defined above can be dosed independently or by use of different fixed combinations with distinguished amounts of the combination partners i.e., simultaneously, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, the invention provides a kit comprising: AN-7 compound, derivatives and/or salts thereof, and an anti-cancer therapy. In some embodiments, the anti-cancer therapy is selected from radiotherapy and chemotherapy. In some embodiments, the anti-cancer therapy comprises a topoisomerase inhibitor. In some embodiments, the anti-cancer therapy comprises doxorubicin.

In one embodiment, it will be appreciated that the compounds/compositions described herein can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies. In some embodiments, the additional active agent comprises a chemotherapeutic agent. In some embodiments, the chemotherapeutic agent is a DNA damaging agent. In some embodiments, the DNA damaging agent is a topoisomerase inhibitor. In some embodiments, the chemotherapeutic agent comprises doxorubicin.

In one embodiment, depending on the severity and responsiveness of the condition to be treated, dosing can be of a single or a plurality of administrations, with course of treatment lasting from several days to several weeks or until cure is effected or diminution of the disease state is achieved.

In some embodiments, the compounds/compositions are administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the compound/composition is the amount of the compound/composition necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

Pharmaceutical compositions containing the presently described AN-7, derivatives, analogues, or salts thereof as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, compositions including the preparation of the present invention formulated in a compatible pharmaceutical carrier are prepared, placed in an appropriate container, and labeled for treatment of an indicated condition.

In one embodiment, compositions of the present invention are presented in a pack or dispenser device, such as an FDA approved kit, which contain one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods of Use

According to some aspects, the present invention provides a method for inducing cell death (e.g., apoptosis), proliferation arrest, or growth arrest in a cell, the method comprises the step of contacting the cell with any of the AN-7 compounds described herein. In some embodiments, the

AN-7 compound described herein reduces or inhibits repair of double strand breaks (DSBs) in DNA in the cell. In some embodiments, the AN-7 compound described herein induces cell death.

In some embodiments, the method further comprises the step of contacting the cell with, or applying thereto an additional anti-cancer therapy (e.g., radiotherapy, chemotherapy, etc.). Each of the steps of contacting may be performed either in vitro, ex vivo, or in vivo. In some embodiments, the cells are contacted with the AN-7 compound and the additional anti-cancer therapy simultaneously, sequentially, or separately.

The terms “cell” and “cells” as used herein encompass cells within a subject's body (in vivo), isolated cells, cell lines (including cells engineered in vitro), any preparation of living tissue, including primary tissue explants and preparations thereof. In some embodiments, the cell is a CTCL cell.

As used herein the term “in vitro” refers to any process that occurs outside a living organism.

The term “in vivo” refers to any process that occurs inside a living organism. The term “ex vivo” refers to a process in which cells are removed from a living organism and are propagated outside the organism.

In another embodiment, the present invention provides a method for enhancing the activity and/or enhancing the efficacy and/or reducing the toxicity of an anti-cancer therapy which induces cell death, the method comprises the step of contacting the cell with at least one of AN-7, a derivative or salt thereof as described herein. In some embodiments, the method further comprises the step of applying or contacting the cell with the anti-cancer therapy.

In some embodiments, contacting the cell with AN-7, a derivative or salt thereof as described herein and applying an additional anti-cancer therapy results in increased efficiency for inducing cell death (e.g., necrosis, apoptosis, or autophagy), proliferation arrest, or growth arrest in a cell compared to each of the AN-7 compounds or the additional anti-cancer therapy when applied by itself. In some embodiments, contacting the cell with the AN-7 compound and an anti-cancer agent results in a synergistic effect.

As used herein, the term “increased efficiency” with reference to an effect refers to a decrease in time for achieving the referenced effect, an increase in the effect (e.g., killing effect) or a combination thereof. In some embodiments, the decrease in time for achieving the effect is at least 1.1, 1.2, 1.3, 1.4, 1.5, 1.6, 1.7, 1.8, 1.9, 2, 3, 4, 5, 6, 7, 8, 9 or 10 fold decrease. In some embodiment, the increase of the effect is increase of cell death in a given time duration. In other embodiments, the increase in the effect may be at least 1.1, 1.2, 1.3, 1.4, 1.5, 1.6, 1.7, 1.8, 1.9, 2, 3, 4, 5, 6, 7, 8, 9 or 10 folds increase in apoptosis in a given time duration. Each possibility represents a separate embodiment of the present invention.

As used herein, the term “synergistic effect” or variant thereof refers to the combined action of two or more agents wherein the combined action is greater than the sum of the actions of each of the agents used alone.

In some embodiments, a synergistic effect of a combination of agents (e.g., AN-7 and an anti-cancer agent) permits the use of lower dosages of one or more of the agents. In some embodiments, a synergistic effect of a combination of agents permits decrease in duration of treatment. In some embodiments, decrease in duration is at least 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 or 10 fold decrease. Each possibility represents a separate embodiment of the present invention.

### Therapeutic Methods

According to some aspects, the present invention provides a method comprising the step of administering to a subject in need thereof a therapeutically effective amount of at least one of AN-7, a derivative, or salt thereof.

In some embodiments, the method is for ameliorating, preventing or treating a cutaneous T-cell lymphoma in a subject in need thereof.

According to some aspects, there is provided a method for treating, ameliorating, reducing and/or preventing a cutaneous T-cell lymphoma in a subject in need thereof, the method comprises the step of: administering to a subject a pharmaceutical composition comprising an effective amount of one or more of AN-7, a derivative or salt thereof (“AN-7 compound”), thereby treating, ameliorating, reducing and/or preventing a cutaneous T-cell lymphoma in a subject in need thereof.

In some embodiments, the method further comprises the step of administering or applying an anti-cancer therapy. In some embodiments, the anti-cancer therapy and the AN-7 compound are administered or applied simultaneously, sequentially or separately.

In some embodiments, the method is for increasing sensitivity of the subject to an anti-cancer therapy. In some embodiments, the method is for improving therapeutic response to an anti-cancer therapy in a subject in need thereof. In some embodiments, the method is for increasing efficacy of the anti-cancer therapy. In some embodiments, the method is for increasing potency of the anti-cancer therapy. In some embodiments, the method is for increasing selectivity of the anti-cancer therapy. In some embodiments, the increase refers to at least 1.5, 2, 3, 4, 5, 6, 7, 8, 9, 10 fold increase in sensitivity, efficacy, potency, selectivity, or any combination thereof, as measured by standard methods well known in the art. Each possibility represents a separate embodiment of the present invention. In some embodiments, the method is for reducing side effects of the anti-cancer therapy. In some embodiments, the increase refers to at least 1.5, 2, 3, 4, 5, 6, 7, 8, 9, 10 fold decrease. Each possibility represents a separate embodiment of the present invention.

As used herein, the term “side effect” or “side effects” refers to the negative symptoms such as nausea, headache, loss of sleep, loss of appetite, and the like, caused by a cancer therapy, as compared to those who have not been treated with the cancer therapy.

In some embodiments, the anti-cancer therapy comprises administration of an anti-cancer agent. In some embodiments, the anti-cancer agent is a DNA damaging agent. In some embodiments, the DNA damaging agent is capable of inducing/mediating double strand breaks (DSBs) in DNA. In some embodiments, the DNA damaging agent is a topoisomerase inhibitor. In some embodiments, the DNA damaging agent is a topoisomerase II inhibitor such as for a non-limiting example, doxorubicin (CAS#: 23214-92-8), etoposide (CAS #: 33419-42-0), mitoxantrone (CAS #: 65271-80-9). In some embodiments, the chemotherapeutic agent comprises at least one topoisomerase II inhibitor selected from: doxorubicin, epirubicin, daunomycin, amscrine, mitoxantrone and derivatives and salts thereof. In some embodiments, the chemotherapeutic agent comprises doxorubicin (CAS #: 23214-92-8) or derivatives or salts thereof.

As used herein, “simultaneously” is used to mean that the two agents (e.g., AN-7 and an anti-cancer agent) are administered concurrently, whereas the term “in combination” is used to mean they are administered, if not simultaneously, then “sequentially” within a timeframe that they both are available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction therebetween, and their respective half-lives. In contrast to “in combination” or “sequentially”, “separately” is used herein to mean that the gap between administering one agent and the other is significant i.e. the first administered agent may no longer be present in the bloodstream in a therapeutically effective amount when the second agent is administered.

As used herein the term “anti-cancer therapy” refers to a therapy useful in treating cancer such as irradiation or radiation therapy or an anti-cancer agent such as but not limited to a pro-apoptotic compound. Non-limiting examples of anti-cancer agents include a chemotherapeutic agent, a hormone, an apoptosis inducer, and an antibody.

As used herein, the term “chemotherapeutic agent” refers to a therapeutic compound and/or drug which may be used to, among other things, treat cancer. The term “chemotherapeutic agent” as used herein encompasses DNA damaging agents, as well as other agents. The term “topoisomerase inhibitor” refers to an agent designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. Non-limiting examples of topoisomerase inhibitors include: doxorubicin, mitomycin C, camptothecin, novobiocin, epirubicin, dactinomycin, etoposide, daunomycin, amscrine, or mitoxantrone. Topoisomerase inhibitors described herein further encompass “analogue(s)” or “derivative(s)” thereof. The terms “analogue(s)” or “derivative(s)” as used herein with reference to a topoisomerase inhibitor, refer to any compound having the activity of a topoisomerase inhibitor which is derived from the known structure of a topoisomerase inhibitor for which the compound is a derivative or an analogue.

As used herein the term “potency” refers to the specific ability or capacity of the anti-cancer therapy (e.g., anti-cancer agent), as indicated by appropriate laboratory tests, to yield a given result.

A high potency refers to the ability to induce a larger response at low concentrations. As used herein, “EC50” is intended to refer to the concentration of a substance (e.g., a protein, a compound or a drug) that is required to induce 50% of the maximum effect (e.g., a biological process). As used herein, “LD50” is intended to refer to the concentration of a substance (e.g., a protein, a compound or a drug) that is required to induce death of 50% of a population of cells.

As used herein, the term “efficacy” refers to the degree to which a desired effect is obtained.

As used herein, the term “selectivity” of a drug with respect to a pharmacological effect refers to the propensity of a drug to preferentially exert the pharmacological effect on a target T-cell towards a treatment goal as opposed to exerting the pharmacological effect on a non-target T-cell towards an undesired side effect of the drug in the non-target T-cell. For a non-limiting example the selectivity of doxorubicin to CTCL cells is increased when co-administered with AN-7.

In some embodiments, the method comprises administering to a subject, simultaneously, sequentially or separately, a therapeutically effective amount of one or more of AN-7, a derivative or salt thereof and doxorubicin.

In some embodiments, there is provided a method for increasing efficacy of anti-cancer treatment in a subject in need thereof, the method comprises the step of co-administering a therapeutically effective amount of one or more of AN-7, a derivative or salt thereof and the anti-cancer agent. In some embodiments, the anti-cancer agent comprises doxorubicin.

In some embodiments, the method comprises administering to a subject, simultaneously, sequentially or separately, a therapeutically effective amount of AN-7 compound and a topoisomerase inhibitor. In some embodiments, the topoisomerase inhibitor is a topoisomerase II inhibitor. In some embodiments, the topoisomerase inhibitor is selected from the group consisting of: doxorubicin (CAS#: 23214-92-8), etoposide (CAS#:33419-42-0), etoposide phosphate (CAS#:117091-64-2), teniposide (CAS#:29767-20-2), epirubicin (56420-45-2), daunomycin (CAS#:20830-81-3), amscrine (CAS#: 51264-14-3) and mitoxantrone (CAS#: 65271-80-9).

In some embodiments, the topoisomerase inhibitor is selected from the group consisting of: doxorubicin, epirubicin, daunomycin, amscrine, and mitoxantrone.

In some embodiments, the method comprises administering to a subject, simultaneously, sequentially or separately, a therapeutically effective amount of AN-7 compound and doxorubicin.

In some embodiments, the term “therapeutically effective amount” is intended to qualify the combined amount of treatments in the combination therapy that will achieve the desired biological response. In some embodiments of the present invention, the desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human. The dosage of the combined agents may be defined in accordance with the dosage clinically used and appropriately selected depending upon factors such as subjects, age and weight of subjects, symptoms, duration of administration, types of formulation, methods of administration, and combination thereof.

In some embodiments, a dose of 0.01-20 mg per kg body weight of AN-7 is administered to a human subject in need thereof. In a non-limiting example, a dose of 0.01-20 mg/kg of AN-7 is administered in soft gel capsules. In another non-limiting example a dose of 0.01-20 mg/kg of AN-7 is administered via IV by infusion for 1 hour.

In some embodiments, AN-7 is co-administered with doxorubicin. In such embodiments, AN-7 may be administered prior to and/or post doxorubicin administration. In some embodiments, AN-7 is administered prior to and post doxorubicin administration.

A person skilled in the art, will appreciate that in conventional treatments doxorubicin is administered via an intravenous (IV) injection through a central line or a peripheral venous line and the drug is given over several min at a dose of 700-1400 mg/kg once a week or once every two weeks (total of 2 treatments/cycle).

In some embodiments, the administered dose of doxorubicin in a combined treatment with AN-7 is the same as in the conventional treatments. In some embodiments, the administered dose of doxorubicin in the combined treatment causes less side effects (e.g., cardiotoxicity) compared to the same administered dose in a conventional treatment.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. As used herein, the terms “subject” or “individual” or “animal” or “patient” or “mammal,” refers to any subject, particularly a mammalian subject, for whom therapy is desired, for example, a human.

In one embodiment, a subject in need thereof is a subject afflicted with and/or at risk of being afflicted with a cutaneous T-cell lymphoma (CTCL). In some embodiments, the cutaneous T-cell lymphoma is selected from Mycosis Fungoides and Sézary syndrome.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, improve the life expectancy of the subject as compared to the untreated state or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compound prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); “Bacteriophage Methods and Protocols”, Volume 1: Isolation, Characterization, and Interactions, all of which are incorporated by reference. Other general references are provided throughout this document.

Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

### Materials and Methods

### Compounds and Reagents

AN-7 was synthesized as described, for example, in U.S. Pat. No. 6,030,961. The following are commercial products: SAHA (Sigma-Aldrich, Rehovot, Israel), doxorubicin hydrochloride (Teva, Petach Tikva, Israel); lymphoprep (Axis Shield, Oslo, Norway); phytohemagglutinin (PHA) (Becton Dickinson, Franklin Lakes, N.J., USA); thiazolyl blue tetrazolium bromide (MTT) reagent (Sigma-Aldrich, Rehovot, Israel); fluorescein isothiocyanate-conjugated annexin V (eBioscience, San Diego, Calif., USA); propidium iodide (PI) (eBioscience), and trypan Blue (Bio-Basic, Unionville, Canada).

### Cell Lines

MyLa 2059 cells, derived from a plaque of a patient with MF, and Hut78 is a homo sapiens cutaneous T lymphocytes cell line, derived from peripheral lymphocytes of a patient with SS. Sezary syndrome Peripheral blood lymphocytes SPBL were derived from 4 patients with SS. All were treatment-naïve patients, and all were diagnosed according to the criteria of the European Organization for Research and Treatment of Cancer (EORTC)-World Health Organization (WHO). In addition, blood samples enriched with NPBL were obtained from leftover blood of 8 healthy blood donors.

### Isolation of Human Peripheral Blood Lymphocytes

Peripheral blood was diluted 1:3 in sterile phosphate-buffered saline (PBS). Lymphoprep was added in the same blood volume with a Pasteur pipette, and the sample was centrifuged. PBL were collected from the white median interphase, rinsed twice with PBS, and suspended in RPMI medium with 10 mM HEPES to 2×106 cells/mL.

### Mossman's Tetrazole Test (MTT)-Based Viability Assay

MyLa cells (10⁴), Hut78 cells (5×10³), SPBL and NPBL (10⁵) were seeded in triplicate in 96-well plates. The PBL were activated with PHA 40 μg/10⁶ cell for 24 h before the experiment. Drugs were added to each well as follows: AN-7, SAHA, Dox, AN-7+Dox, SAHA+Dox. The cells were then placed in a humidified incubator for 72 h. The ratios of HDACI to Dox in the combined-treatment experiments were based on the ratio of the IC₅₀ of each drug alone. The MTT reagent was added to a final concentration of 0.5 mg/mL, and the cells were incubated for an additional 4 h. Thereafter, 0.1N HCl in isopropanol was added, and cell viability was determined using an ELISA reader (PowerWaveX, BioTek, Winooski, Vt., USA) at a 570 nM wavelength with background subtraction at 630-690 nM.

### Trypan-Blue-Based Viability Assay

MyLa cells (2×10⁵ cells/mL), Hut78 cells (2×10⁵ cells/mL), or NPBL (1×10⁶ cells/mL (after overnight incubation with PHA) were treated with an HDACI under two conditions: long exposure—24 h for MyLa cells and Hut78 cells and 48 h for PBL; or short exposure—4 h incubation followed by washout and re-incubation with new medium for another 44 h (MyLa) or 20 h (Hut78). All samples were diluted 1:5 with trypan blue (0.5%), and unstained (viable) cells were counted under a light microscope.

FACS Analysis with Annexin V and Propidium Iodide Staining

MyLa and Hut78 cells (2×10⁵ cells/mL) were exposed to SAHA or AN-7 as in the trypan-blue assay. The cells (2.5×10⁶ cells/mL) were washed in PBS and binding buffer and were resuspended in binding buffer, and of fluorescein isothiocyanate-conjugated annexin V (5 μL) was added to a 100 μL cell suspension for 10-15 min. Incubation was performed at room temperature under light-protected conditions. The cells were then washed in binding buffer and were resuspended in the binding buffer and propidium iodide (PI) (5 μL) was added. The samples were analyzed by flow cytometry (FACS Calibur 4.1.6, Becton Dickinson): fluorescein-labeled annexin V at a 530 nm wavelength, and PI at a 585 nm wavelength. The percentage of cells was calculated by their distribution in a fluorescence dot plot generated with FCS Express 4 software (De Novo Software, Los Angeles, Calif., USA). Early (annexin V-positive) and late (annexin V+PI-positive) apoptotic cells were summed to yield the total number of apoptotic cells.

### Nuclear Fractionation for Histone Detection

Cells (5×10⁶) were suspended in 300 μL of cytoplasmic buffer (HEPES 10 mM, KCl 10 mM, EDTA 1 mM, EGTA 1 mM, DTT 1 mM). After 20 min of incubation on ice, the mixture was passed 5 times through a 25-G syringe and then centrifuged briefly to obtain the cytoplasmic supernatant.

The nuclear pellet was suspended in 40-60 μL of nuclear buffer (cytoplasmic buffer+10% glycerol), incubated with shaking at 4° C. for 15 min, and centrifuged. The supernatant was collected as a nuclear fraction.

### Western Blot Analysis

Cell extracts were separated by SDS-polyacrylamide gel electrophoresis (SDS-PAGE), transferred to a nitrocellulose membrane, and subjected to immunoblot with primary and secondary antibodies, as listed in table 1.

TABLE 1
Primary and Secondary Antibodies Used for Western Blot (WB)

| Type | Reactivity (isotype) | Host | Dilution for WB | Manufacturer |
|---|---|---|---|---|
| Primary/monoclonal | Anti-cleaved caspase-3 (IgG) | Rabbit | 1:1000 | Cell-signaling |
| Primary/polyclonal | Anti-PARP-Poly-ADP-ribose | Rabbit | 1:1000 | Cell-signaling |
| | polymerase 3 (IgG) | | | |
| Primary/polyclonal | Anti-p21-cyclin-dependent | Rabbit | 1:200 | Santa Cruz |
| | kinase inhibitor 1A (IgG) | | | |
| Primary/polyclonal | Anti-Bax-BCL2-associated X | Rabbit | 1:1000 | Abcam |
| | protein (IgG) | | | |
| Primary/polyclonal | Anti-acetylated N-terminus of | Rabbit | 1:500 | Millipore |
| | histone H3 (IgG) | | | |
| Primary/polyclonal | Anti- histone deacetylase 1 | Rabbit | 1:2000 | Sigma |
| | (HDAC1) (IgG) | | | |
| Primary/monoclonal | Anti-actin (IgG) | Mouse | 1:8000 | Molecular probe |
| Secondary/polyclonal | Anti-rabbit IgG (H + L) | Goat | 1:5000 | LI-COR Biosciences |
| Secondary/polyclonal | Anti-mouse IgG (H + L) | Goat | 1:5000 | LI-COR Biosciences |

### Computational and Statistical Analysis

Viability and apoptosis curves were based on the averages of at least 3 independent experiments. The standard error (SE) was calculated for each group as follows: SE=standard deviation/√n, where n is number of values in the group. The average drug concentrations causing a 50% reduction in cell viability, IC₅₀, were determined with the formula for linear or polynomial regression derived from the best-fitted curve of percent viability versus drug concentrations (≧3 independent dose-response titrations). The selective toxicity index (SI) was calculated as follows: SI=IC₅₀ NPBL/IC₅₀ MF/SS cells, where SI>1 indicates toxic selectivity to MF/SS cell lines, SI<1 indicates toxic selectivity to NPBL, and SI=1 indicates no selectivity. Significant differences in selectivity among groups were analyzed by comparing the IC₅₀ values using two-tailed unpaired t-test using Excel or by comparing the differential effects for all the dose response titrations using ANOVA with repeated measures.

For analysis of drug interactions, drug concentration-dependence plots for each drug alone and in combination were formulated, and the combination index (CI) was calculated using CompuSyn software (ComboSyn, Inc. Paramus, N.J., USA), where CI>1 indicates an antagonist interaction between two drugs, CI=1 indicates an additive interaction, and CI<1 a synergistic interaction.

### Comet Assay—a Single-Cell Gel Electrophoresis

Comet assay using the Trevigen Comet assay kit (Trevigen Inc., Gaithersburg, Md., USA) was done according to the manufacturer's protocol. MyLa or Hut78 cells were treated with drugs, washed, incubated for interval time, washed in PBS, counted and suspended in PBS. 1 ×10⁵ cells/ml were incubated with pre-heated low melting agarose for 20 min in 37° C., loaded on a comet-slide and kept in the dark for 30 min at 4° C., in cold lysis solution for another 20 min, and finally for 20 min with unwinding solution in RT. The slides were run with cold fresh alkaline electrophoresis buffer in the COMET-20 electrophoresis system (Scie-Plas, Harvard apparatus, UK) for 20 min in 21 volts, 4° C. Slides were stirred gently and washed twice in dH20 bath for 5 min and in 70% ethanol for 5 min. On the day of the analysis, the slides were stained with SYBR Gold (1:10,000 dilutions in TE buffer, pH 7.5) for 10 min in 4° C. protected from light, and dried in RT for at least 30 min. The cells were scored under fluorescence microscope (Nikon eclipse 55i, Tokyo, Japan) at a magnification ×10. Cell scores were analyzed using the software “Comet Assay IV” (perceptive instruments, UK). The relative length and intensity of DNA tails relative to heads is proportional to the amount of DNA damage in individual nuclei, define as “Tail Moment”. 24h after treating the cells the mean tail moment reaches its maximal level, therefore the percent of repaired damage was calculated as follows: (mean tail moment at 24h)−(mean tail moment at indicated time)/mean tail moment at 24 h. At least a total of 100 cells were measured per time-point.

### Western Blotting

Cell fraction—3×10⁶cells were suspended in 100 μl hypotonic buffer (HEPES 10 mM, KCl 5 mM, MgCl₂ 1.5 mM, DTT 1 mM, NP-40 1%), incubated for 15 min in ice, centrifuged and the supernatant fraction was subjected to western blot of pKAP1 and actin. The sediment was washed once with hypotonic buffer followed by suspension in 100 μl of hypertonic buffer (HEPES 10 mM, NaCl 500 mM, MgCl₂ 1.5 mM, DTT 1 mM, NP-40 1%), incubated for 15 min in ice, centrifuged, supernatant was removed and the sediment was boiled, vortexed for 10 min at 95° C. with sample buffer ×3 for 3 times and subjected to western blot of γH2AX.

Whole cells extract—Cells were suspended in lysis buffer (Tris-HCl 50 mM, NaCl 150 mM, NP-40 0.5%), incubated for lh on ice, centrifuged and the supernatant was collected. Protease and Phosphatase inhibitors were added to all buffers at a 1:100 dilutions. 30 μg of protein cell extracts were separated by SDS-polyacrylamide gel electrophoresis (SDS-PAGE), transferred to a nitrocellulose membrane, and subjected to immunoblot with the following antibodies: NBS1, Novus Biologicals, NB100-143 1:1000; γ-H2AX, Bethyl, A300-081A 1:2500; pS 824 KAP1, Bethyl, A300-767A 1:25,000; ku70, Abcam, ab83501 1:5000; DNA-PK, Santa Cruz, NA-57 1:10,000; Rad51, Santa Cruz, sc-8349, 1:200; MRE11, Santa Cruz, sc-58591:100 ; Actin MP-69100, Molecular Probe, 1:8000, Goat anti-rabbit IRDye 680cw, LI-COR Biosciences 1:5000; Goat anti-mouse IRDye 800cw, LI-COR Biosciences 1:5000; Donkey anti-goat IgG-HRP sc-2020, Santa Cruz Biotechnology 1:5000. Bands were visualized by Odyssey Infrared Imaging System and the bands intense were normalized to actin.

### Immunofluorescence

MyLa or Hut78 cells were treated with drugs, washed, incubated for interval time, and then 2×10⁵cells/m1 were centrifuged with cyto-slides (CytoSlide, Shandon, Thermo, Ma, USA) in CYTOSPIN 4 (Shandon, Thermo scientific, West Palm Beach, USA) for 5 min 1000 rpm. Slides were washed with cold PBS on ice, pre-extracted with cold PBS-NP40-0.2% for 10 min, washed 3 times with cold PBS, fixed with 4% Paraformaldehyde for 10 min at RT, and washed 3 times with PBS. Slides were incubated with anti γ-H2AX, Bethyl A300-081A 1:1000, for 1.5 h in RT, washed twice in PBS-T (0.05% Tween in PBS) and once with PBS, incubated with Donkey anti rabbit IgG Alexa Fluor A-21206 1:700, for 30 min at RT, washed 3 times with PBS, dried gently, covered with 1 drop of Fluorescent mounting medium with DAPI (GBI-labs, WA, USA) and on top with cover glass. Slides were visualized in a light microscope (Olympus BX52, Tokyo, Japan). Foci analysis was performed using ImageJ software and PZfociEZ plugin. % of cells with >5 foci/cell was considered as damaged cells. % of repaired damage was calculated as follows: (% of cells with >5 foci/cell at 3 h)−(% of cells with >5 foci/cell at indicated time)/(% of cells with >5 foci/cell at 3 h). At least a total of 150 cells were measured per time-point.

### HR Activity Assay

U2OS cells with the linearized HRsub reporter (Puget et al. DNA Repair 4 (2005) 149-161) were transfected with: SceI-expression vector pCBASce; pCBA vector without Sce-I; and GFP control vector, each one separately with FuGENE 6 transfection reagent (Promega). 24 h later 2×10̂5 cells/ml were seeded in 35 mm plates and 6 h later were treated with 1 mM AN-7 for 48 hr. Cells were washed with PBS then harvested with Trypsin solution, neutralized with PBS+10% FBS, centrifuged in 1200 rpm for 10 min, suspended with PBS+10% FBS and GFP-positive cells were quantified by flow cytometry analysis. Cells excitation was done with 488nm laser; the reading of FL1 values was done in 530 nm. Percent of cells with repaired DSB induced by Sce-I via HR are presented as percent of GFP positive cells which are calculated as follow: (%GFP of cells expressing Sce-I pCBASce)−(%GFP of cells expressing empty pCBASce)/(%GFP of cells expressing GFP control vector).

Statistical analysis—The significance of the differential effects among the comparative groups was determined by a two-tailed, un-paired, t-test, using Excel software.

### Example 1

### AN-7 is More Effective and Selective in MF/SS Cell Lines and SPBL than SAHA

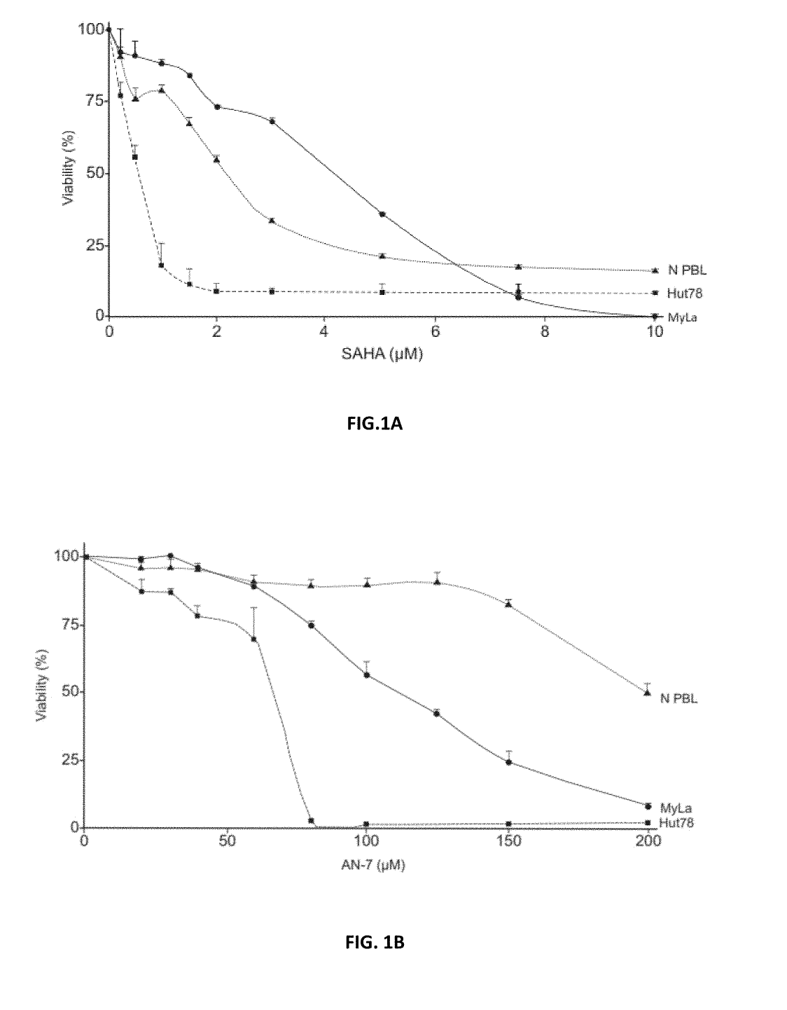

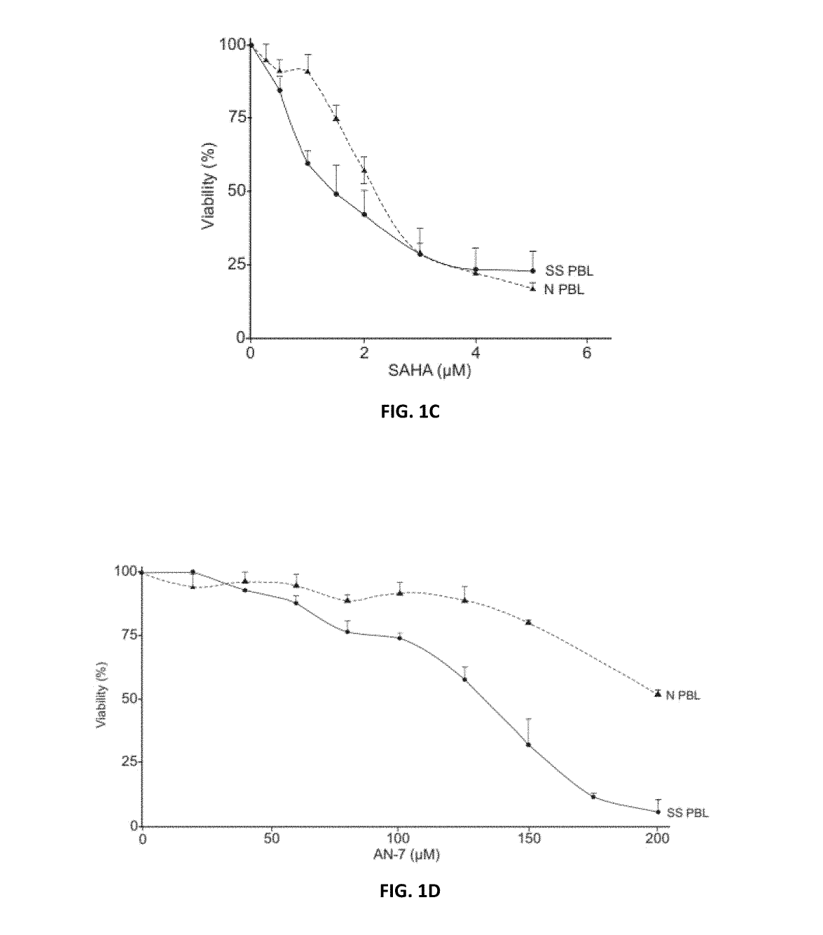

Dose-effect viability curves derived from the MTT-based assay showed that SAHA and AN-7 were toxic to both MyLa cells and Hut78 cells (FIGS. 1A and 1B). Comparison by dose-response titration (ANOVA with repeated measures) showed that SAHA was significantly selective to Hut78 cells (p=1.7×10⁻⁵) and significantly nonselective to MyLa cells (p=0.168) whereas AN-7 was significantly selective to both cell types (p=1×10⁻⁵ and p=2.89×10⁻⁴, respectively) (FIG. 1E). Comparison by IC₅₀ values yielded similar results. In the presence of high doses of AN-7, which were lethal to Hut78 and MyLa cells, 50% of the NPBL survived. By contrast, high doses of SAHA were lethal to all cells (FIGS. 1A and 1B).

To confirm the in vitro results, the toxicity and selectivity of AN-7 and SAHA in SPBL were tested (FIGS. 1C and 1D). Analysis by the IC₅₀ values derived from the viability curves showed that

AN-7 induced selective death but SAHA induced nonselective death. Results were similar on comparison by dose-response titration (p<0.001 and p=0.5173, respectively). High doses of AN-7 were more lethal to SPBL than high doses of SAHA (FIGS. 1C and 1D).

Table 2. shows the IC₅₀ and SI values of SAHA and AN-7 in MF/SS cell lines and SPBL and NPBL based on the viability curves of FIGS. 1A-D, and their p values.

TABLE 2
IC₅₀ and SI values following treatment with AN-7 or SAHA

| | AN-7 | | | SAHA | | |
|---|---|---|---|---|---|---|
| | IC₅₀ | p value vs | Selectivity | IC₅₀ | p value vs | Selectivity |
| Cell type | (μM) | normal PBL | index (SI) | (μM) | normal PBL | index (SI) |
| Myla | 120 ± 5.3 | 0.0001 | 1.7 | 4.3 ± 0.18 | 0.0005 | 0.5 |
| Hut78 | 59 ± 4.3 | 2*10 − 5 | 3.4 | 0.7 ± 0.05 | 0.0002 | 3 |
| Sezary PBL | 128 ± 5.2 | 0.00097 | 3.56 | 2.96 ± 0.74 | 0.583 | 1.21 |
| Normal PBL | 200.3 ± 2.8 | | | 2.1 ± 0.1 | | |

### Example 2

### AN-7 has a More Rapid and Longer Lasting Toxic and Apoptotic Effect on MF/SS Cell Lines than SAHA and Induces Stronger Apoptosis in SPBL

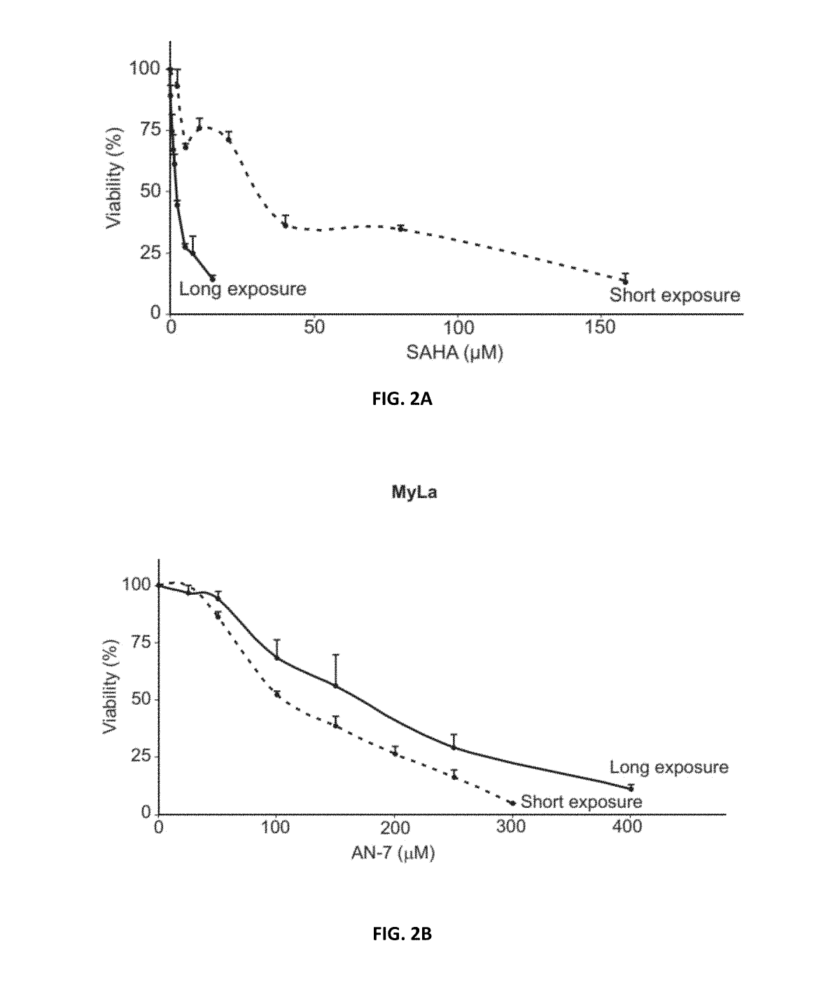

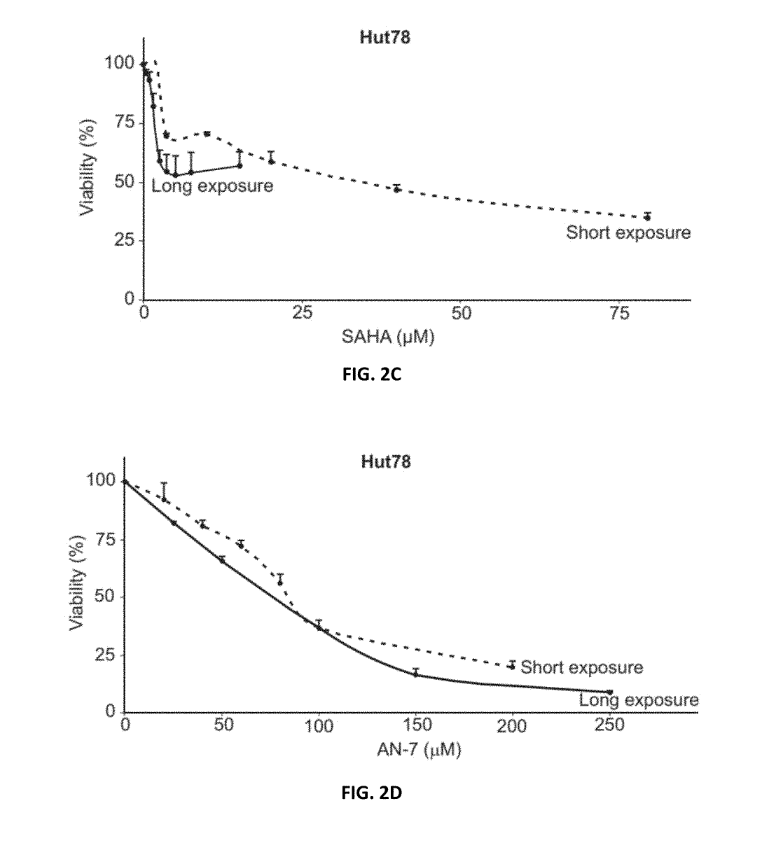

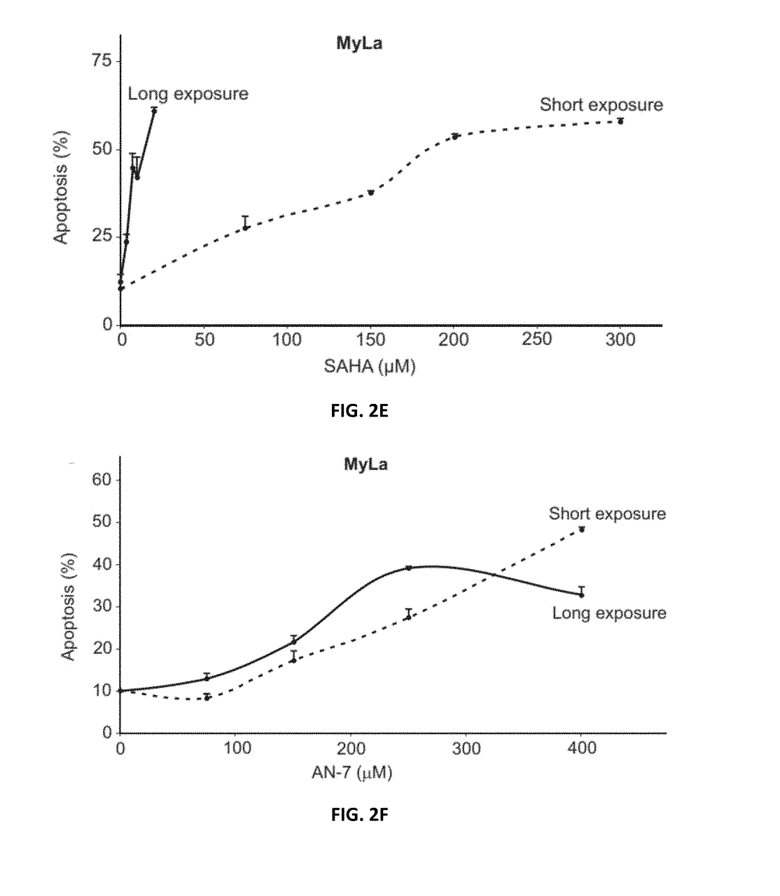

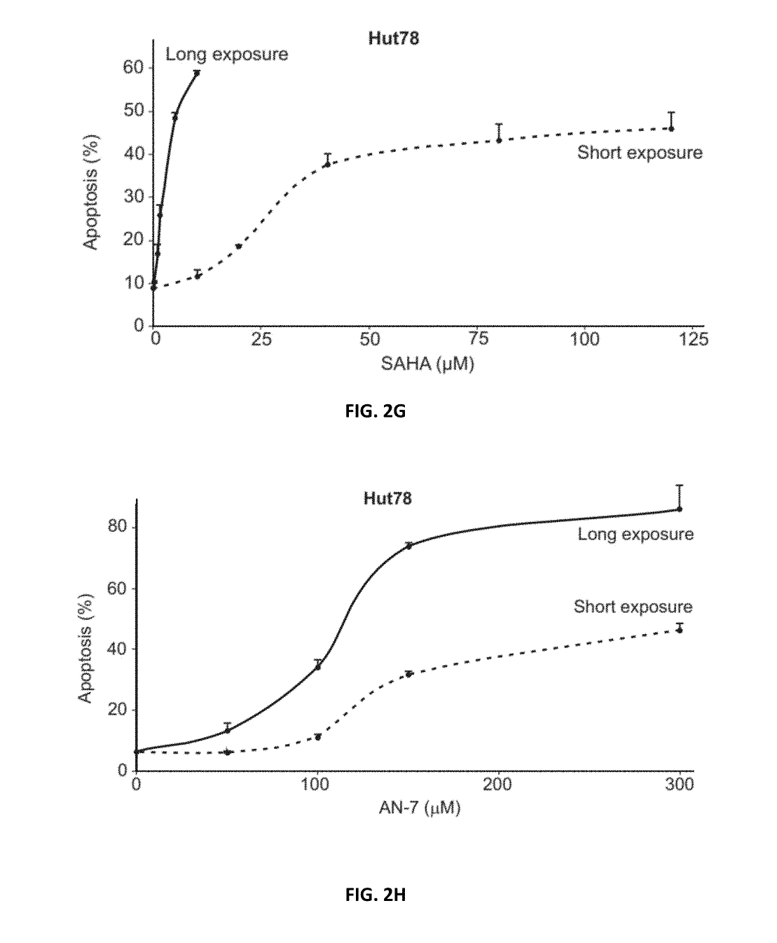

The sensitivity of the MF/SS cell lines to AN-7 and SAHA after long or short exposure, was tested using trypan blue staining (FIG. 2A-2D, Table 3). Table 3 summarizes the IC₅₀ values of short and long exposure to SAHA and AN-7 in MF/SS cell lines based on the viability curves of FIGS. 2A-D. In MyLa cells treated with SAHA, the IC₅₀ of short exposure was 14.3-fold higher than the IC₅₀ of long exposure (p=0.0012); in Hut78 cells, the IC₅₀ of short exposure was 17.1-fold higher than for long exposure (p=2.28×10⁻⁶). By contrast, there was no difference in AN-7 toxicity in MyLa cells by length of exposure (p=0.644), and only a minor difference (0.88-fold) in Hut78 cells (p=0.017).

TABLE 3
IC₅₀ and SI values following short and long exposure to AN-7 or SAHA

| | IC₅₀ (μM) | | | | | |
|---|---|---|---|---|---|---|
| | AN-7 | | | SAHA | | |
| HDACI | Long | Short | IC₅₀ short exposure/ | Long | Short | IC₅₀ short exposure/ |
| compound | exposure | exposure | IC₅₀ long exposure | exposure | exposure | IC₅₀ long exposure |
| SAHA | 2.3 ± 0.08 | 33 ± 3.7 | 14.3 | 2.4 ± 0.08 | 41 ± 1 | 17.1 |
| AN-7 | 159 ± 34.8 | 141 ± 9.8 | 0.88 | 67 ± 2.5 | 88 ± 4.6 | 1.3 |

To test the apoptotic effect of the two HDACIs by length of exposure, different lengths of exposure were used and analyzed for their annexin V and PI staining. Both AN-7 and SAHA induced apoptosis in the MF/SS cell lines (FIG. 2E-2H). For AN-7, there was no significant difference in the degree of apoptosis by time of exposure in either cell line (MyLap=0.9, Hut78 p=0.25). However, for SAHA, short exposure was associated with 2.75-fold less apoptosis in MyLa cells (p=0.034) and 2.5-fold less apoptosis in Hut78 cells (p=0.046) compared to long exposure.

Subsequently, the apoptosis inductions of AN-7 and SAHA on two PBL samples of SS patients were tested ex vivo. The drugs' concentrations for apoptosis induction were determined based on the average IC₅₀ of each drug in SPBL (for AN-7, 128 μM and for SAHA, 2.96 μM, FIG. 1E), using doses of about 1.5 folds higher than the IC₅₀′ s for incubation of 48 h instead of 72 h that were used in the viability assay. To this end, PBL from 2 SS patients were plated at a concentration of 0.5×10⁶ cells/mL, and were then treated with SAHA 4 μM or AN-7 200 μM for 48 h. The cells were then stained with annexin V and PI and analysed by FACS.

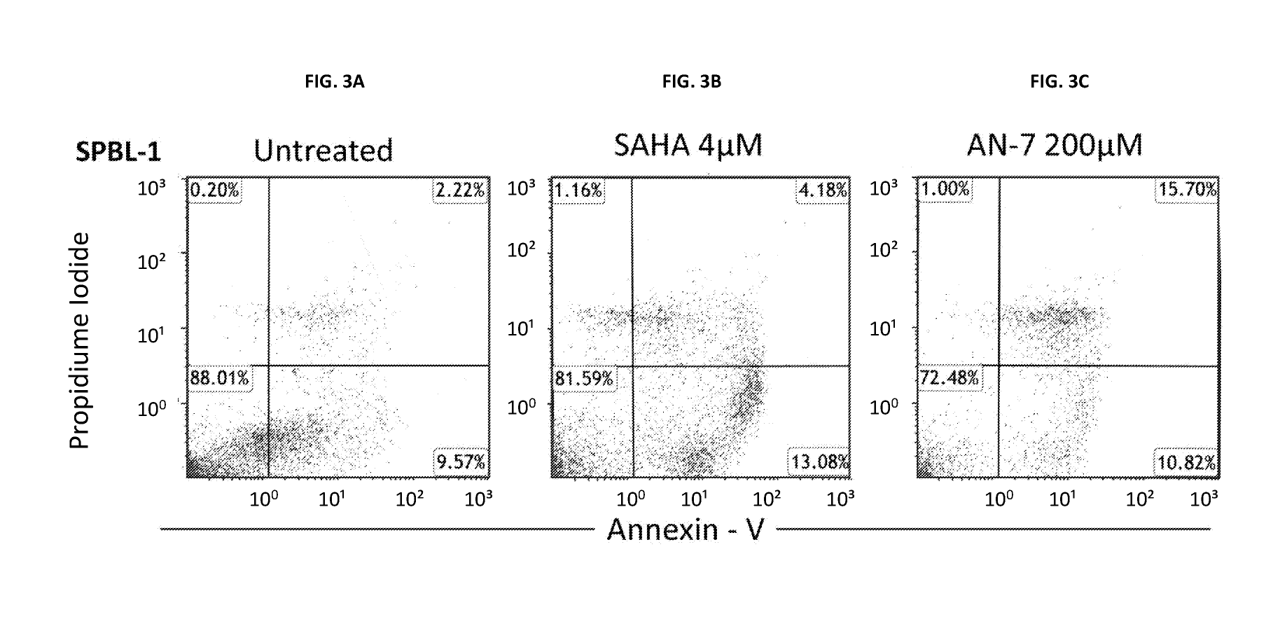

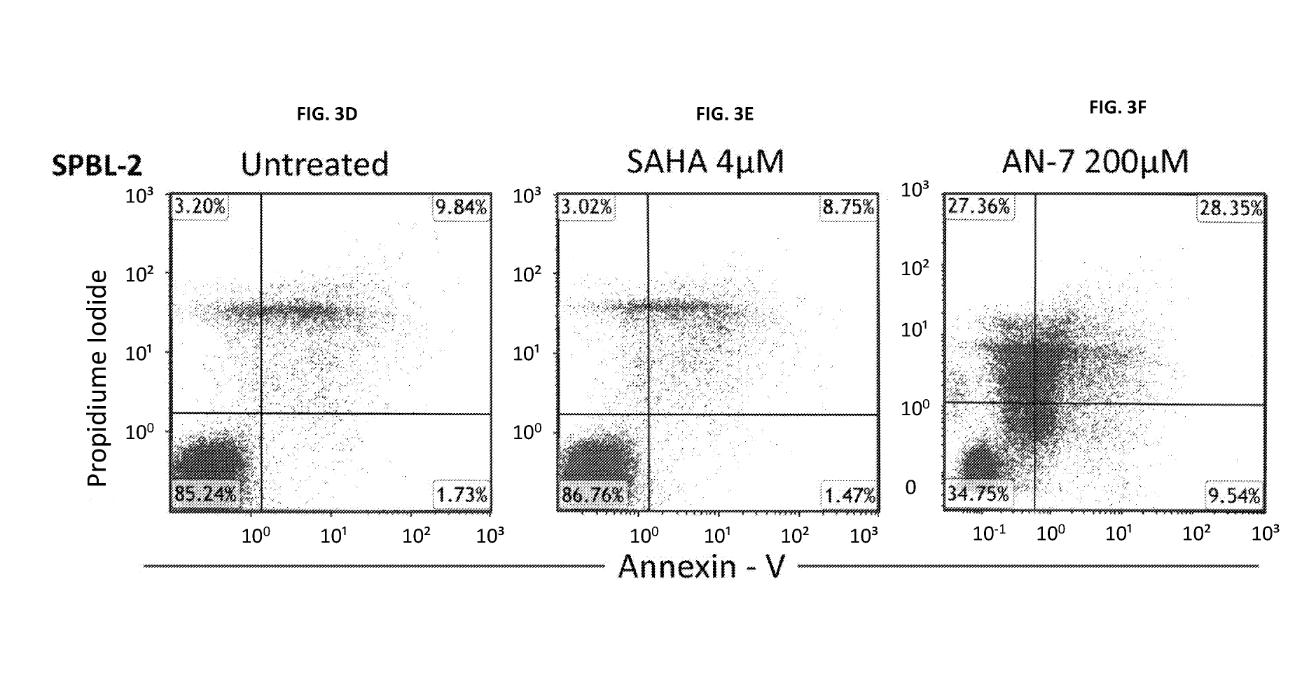

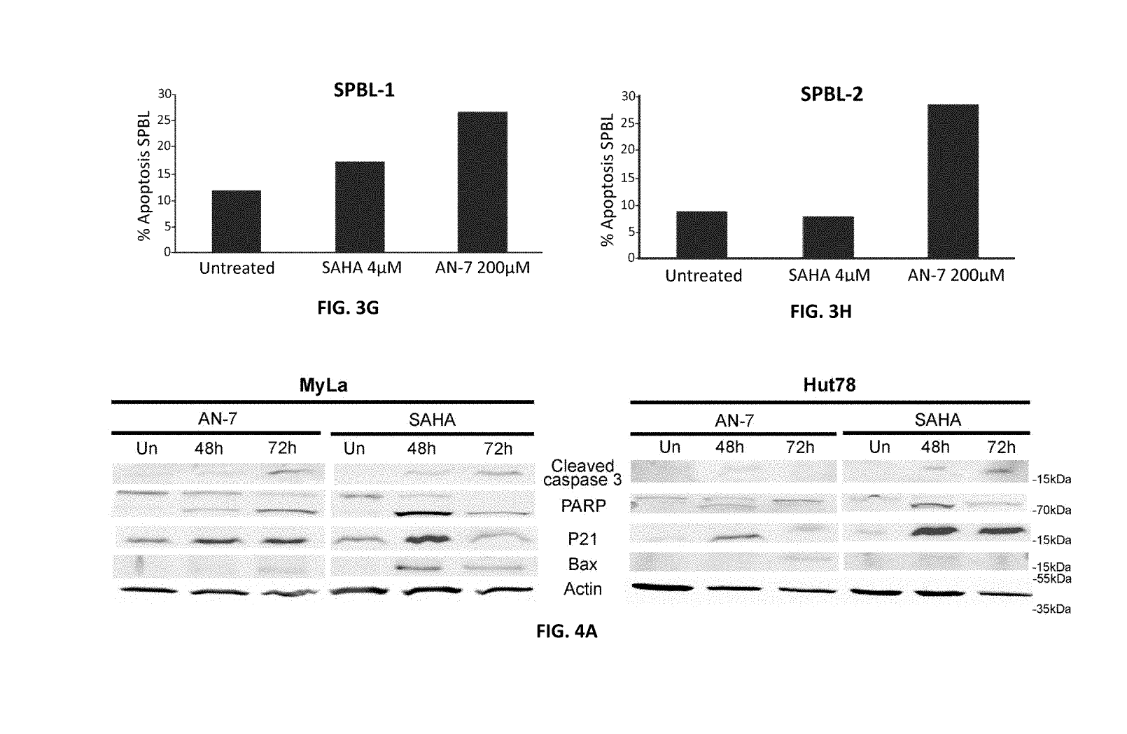

AN-7 directed more SPBL cells into apoptotic death than SAHA, and in one SPBL sample it induced also stronger necrosis (FIGS. 3A-H).

The apoptosis and viability results demonstrated that AN-7 works faster than SAHA and is highly effective and selective after both short and continuous treatment. For SAHA to achieve a maximum apoptotic effect, it needed to be present for a longer time than AN-7.

### Example 3

### AN-7 and SAHA Induce the Expression of Proapoptotic Proteins, Downregulate HDACI Expression and Upregulate Acetylation of Histone 3 (H3) in MF/SS Cell Lines

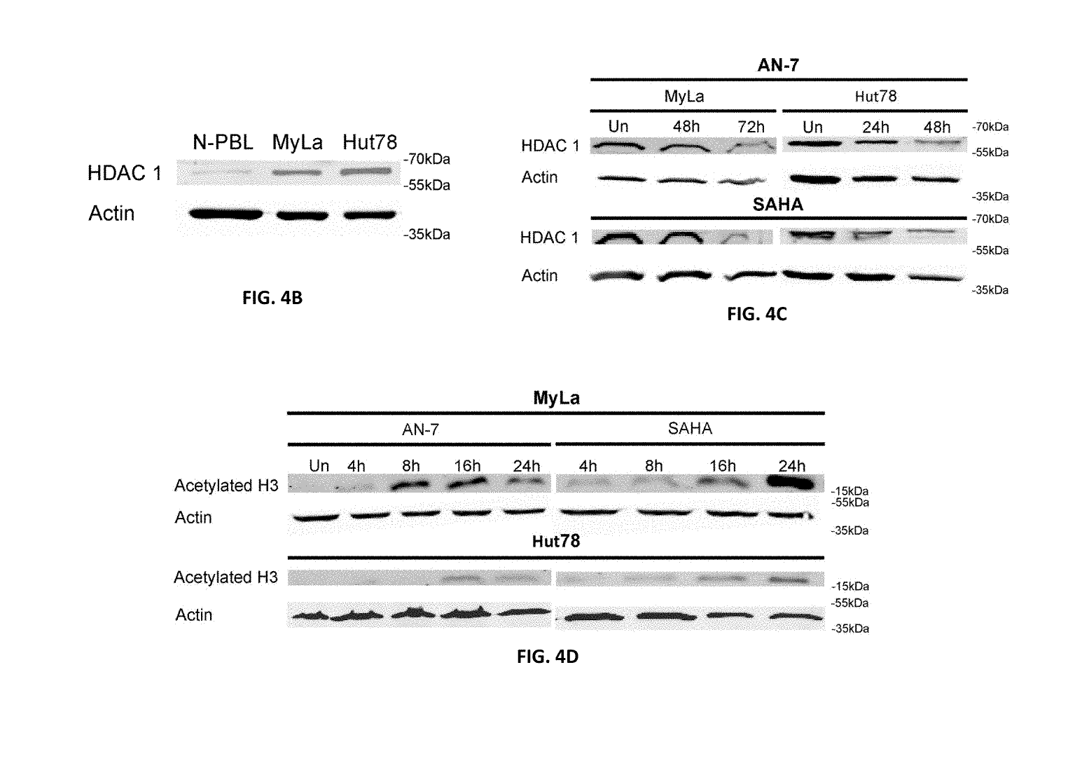

To characterize the mechanism underlying HDACI-induced apoptosis, Western blot analysis was used to measure the levels of several pro-apoptotic proteins in MF/SS cell lines treated with AN-7 or SAHA at concentrations previously shown to cause about 60% apoptosis (FIG. 4A). Both SAHA and AN-7 treatment led to cleavage of caspase 3 and poly ADP-ribose polymerase (PARP) and the production of P21 and Bax in both cell lines. However, there was a stronger signal in response to SAHA.

Studies have shown that neoplasia, including lymphoid and myeloid leukemia, is associated with abnormalities in the expression, function, or recruitment of HDAC and/or its counterpart, histone acetyl transferase (HAT). It was found that both MF/SS cell lines expressed high levels of HDACI compared to NPBL (FIG. 4B) and that these levels were downregulated on exposure to either AN-7 or SAHA (FIG. 4C). Thus, SAHA and AN-7 apparently inhibit the activity of HDAC enzymes and thereafter influence their expression level resulting in prolonged acetylation of histone and non-histone proteins. The expression of acetylated H3, a direct substrate of HDACIs, was induced by both drugs, with earlier AN-7-mediated kinetics in MyLa cells (FIG. 4D). This result suggests a more rapid action of AN-7 in inhibiting HDAC activity.

### Example 4

### AN-7 Acts Synergistically with Dox and SAHA Acts Antagonistically with Dox in MF/SS Cell Lines and SPBL

In order to evaluate the toxicity of HDACI and Dox an MTT assay of MyLa cells, Hut78 cells, and SPBL treated for 72 h with drug combinations, in comparison to NPBL, was performed. The combination ratio between the HDACIs and Dox were based on the ratio between their IC₅₀ values for each cell type, as follows: MyLa cells were treated with Dox+AN-7, 1:3000 (molar ratio) and with Dox+SAHA1:150 (molar ratio). Hut78 cells were treated with Dox+AN-7, 1:2600 (molar ratio) and Dox+SAHA, 1:38 (molar ratio). SPBL were treated with Dox+AN-7, 1:1781 (molar ratio) and Dox+SAHA, 1:20 (molar ratio). NPBL were treated at same molar ratio as SPBL.

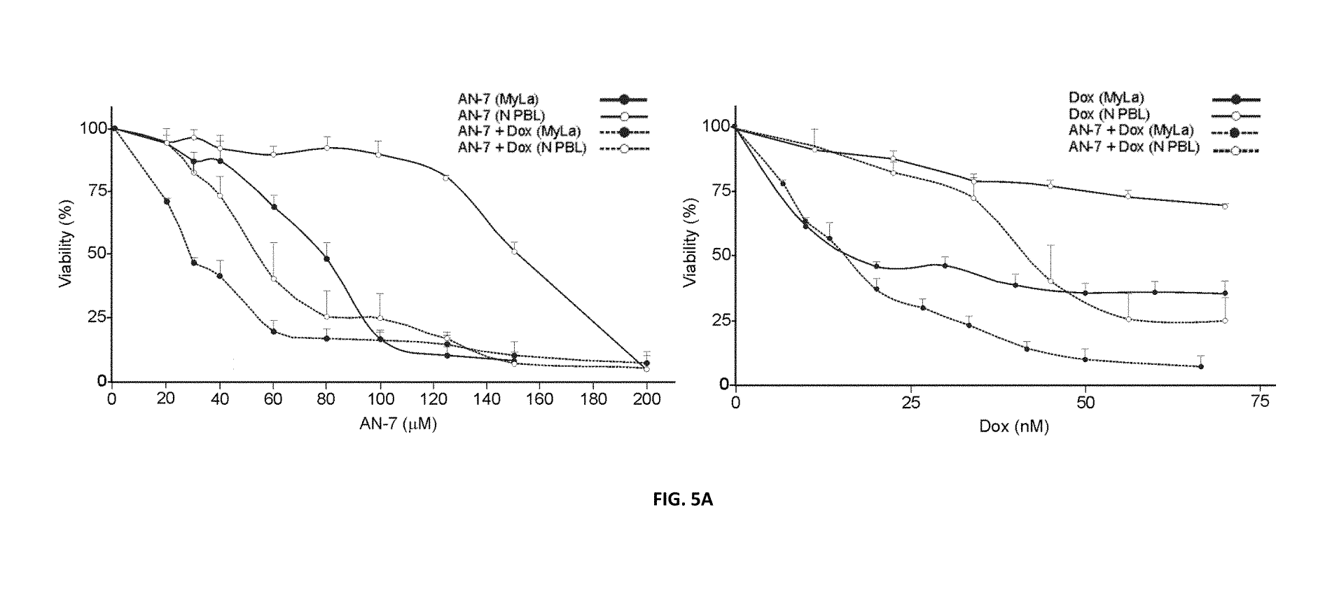

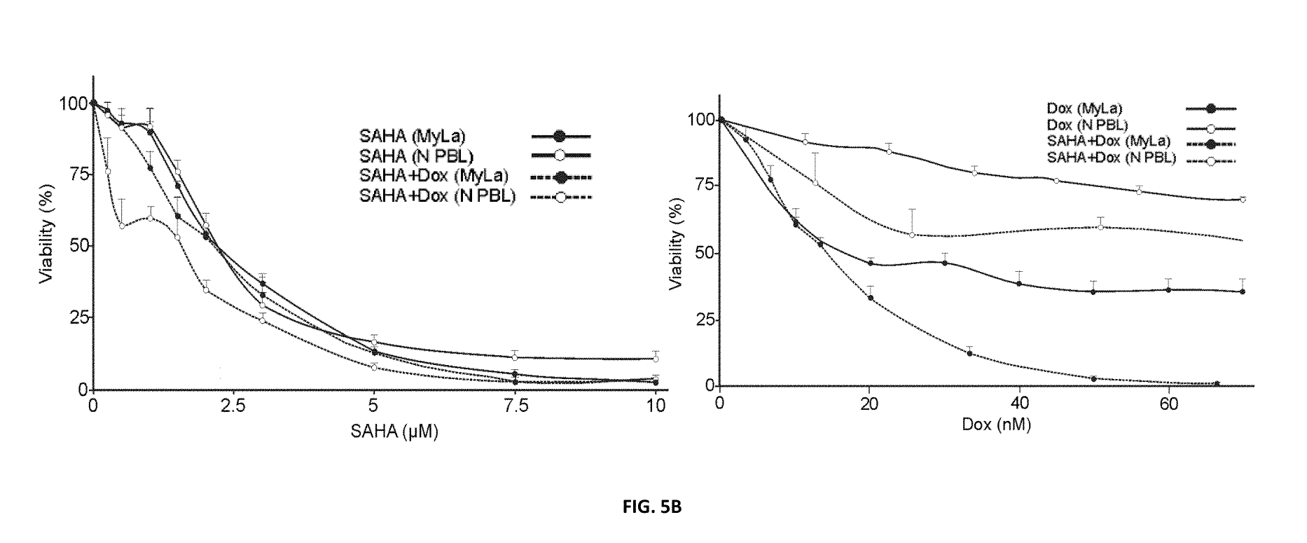

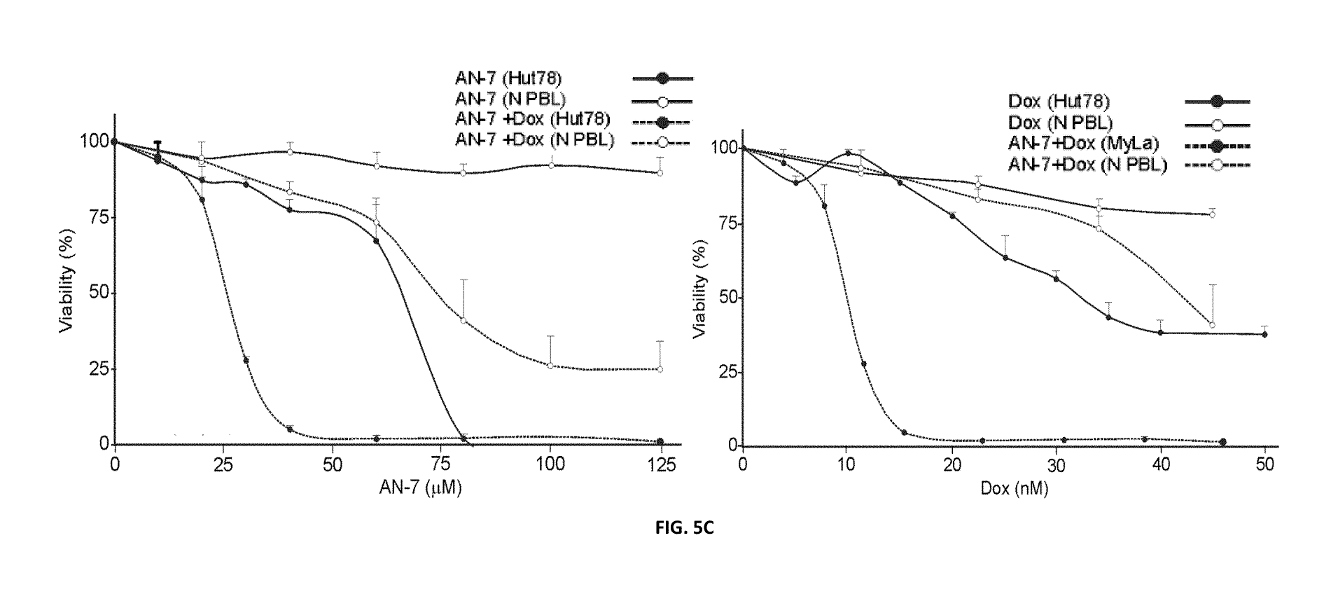

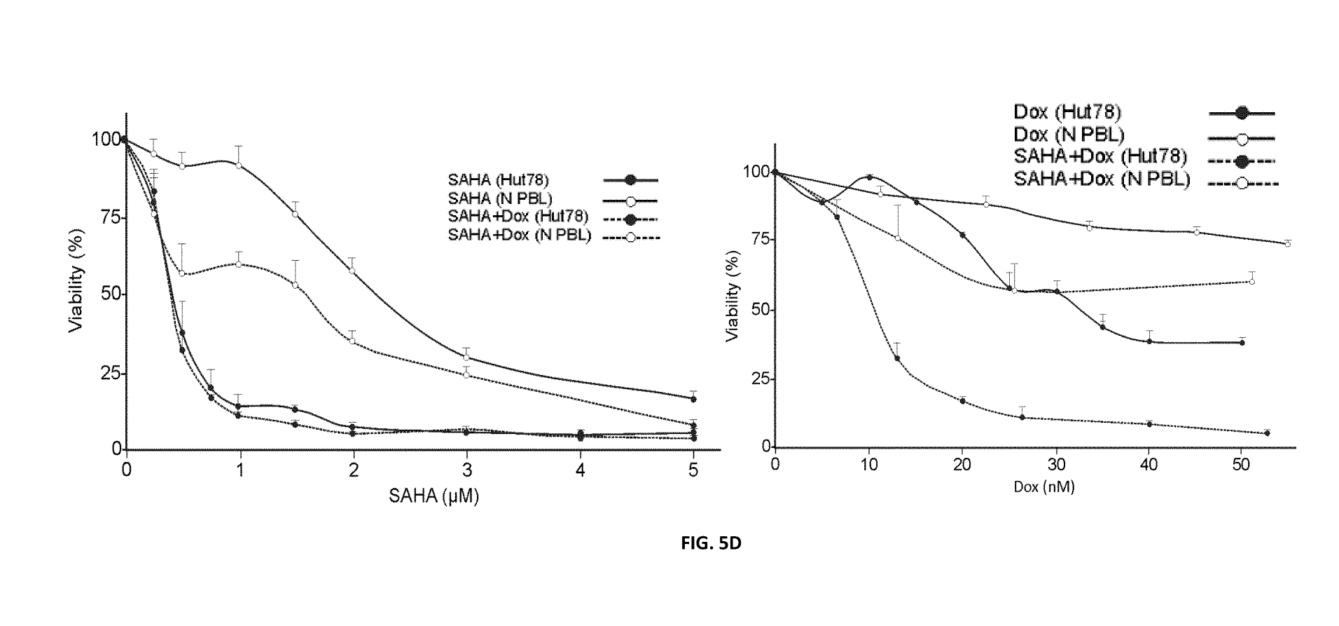

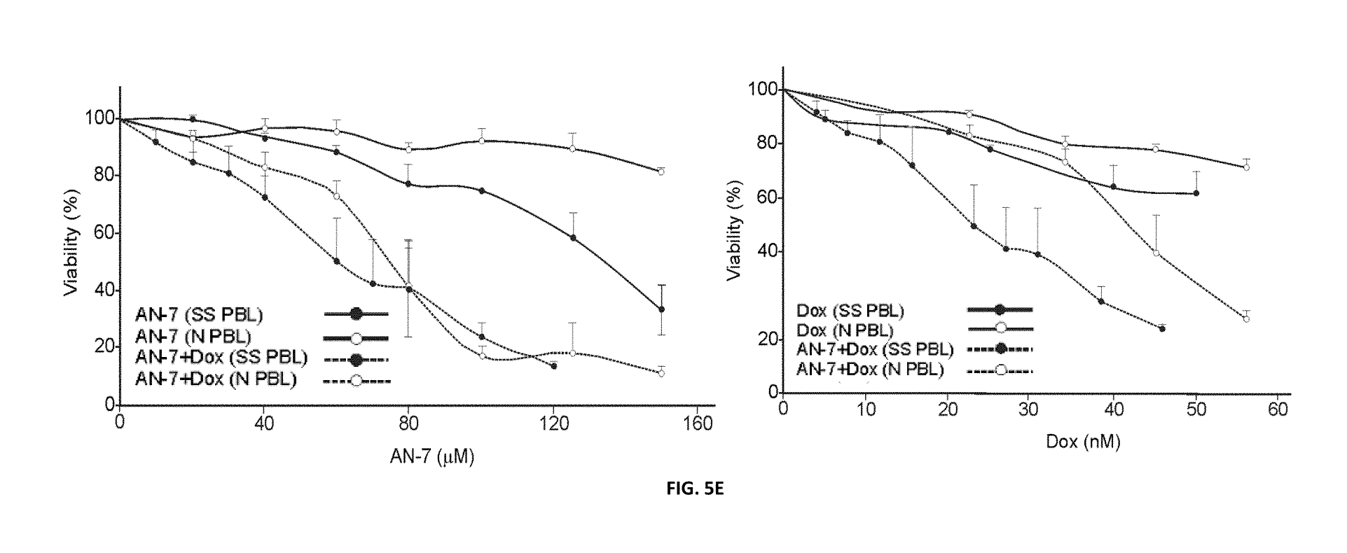

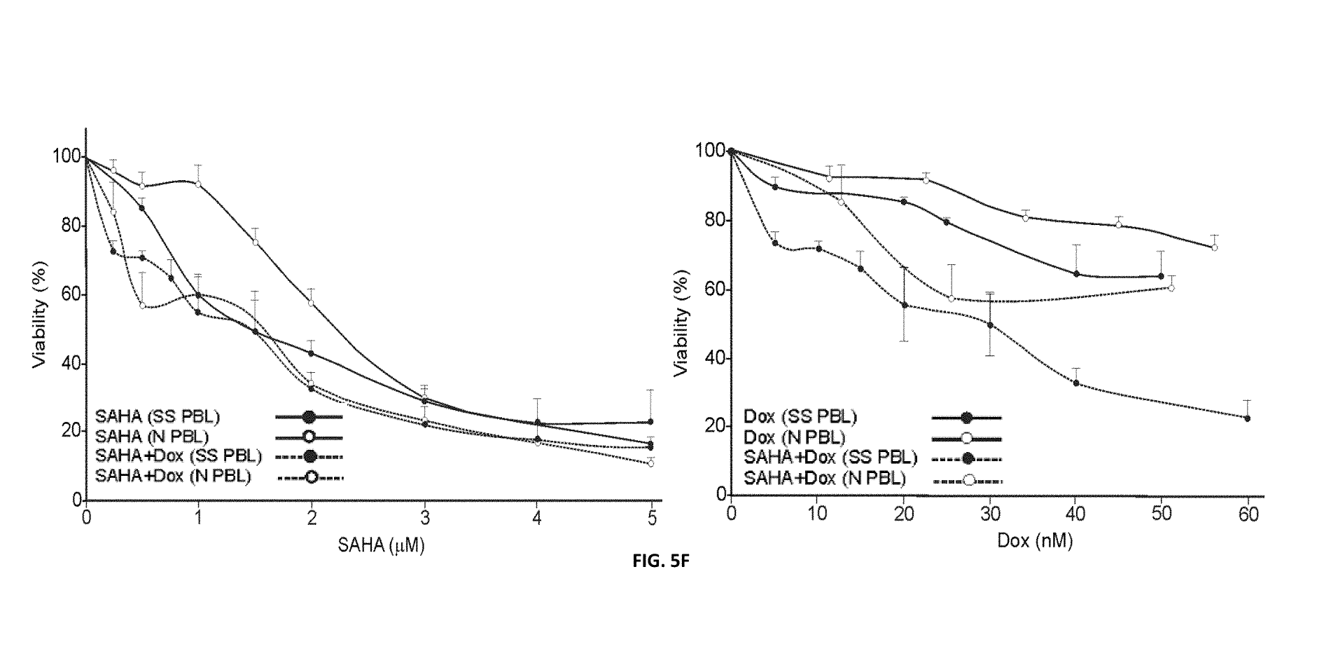

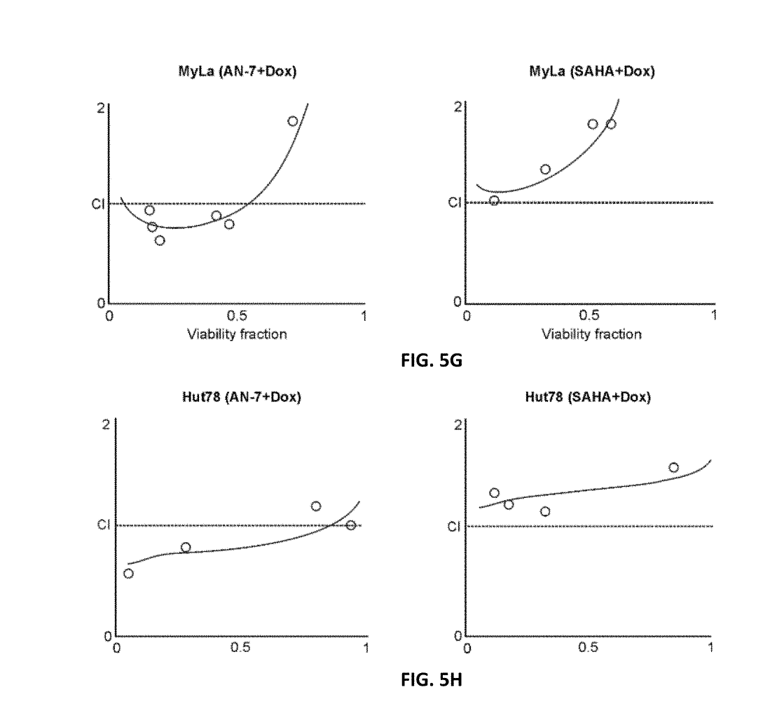

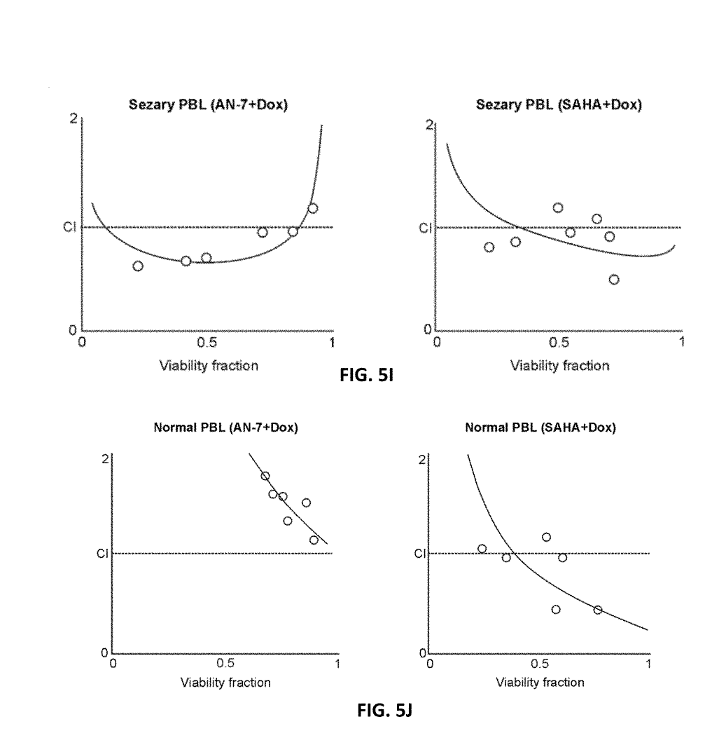

MTT viability assay analysis of the anti-cancer effect and selectivity of HDACIs combined with Dox in MF/SS cell lines and SPBL compared to NPBL revealed a dramatic reduction in the IC₅₀ of each drug in the AN-7+Dox combination (p=0.0002 in MyLa cells, p=0.003 in Hut78 cells, p=0.054 in SPBL) but not in the SAHA+Dox combination (p=0.8,p=0.3, and p=0.424, respectively) (FIGS. 5A-5F).

Table 4 summarizes the IC₅₀ values, derived from the viability curves of FIGS. 5A-F, of AN-7, SAHA alone or combined with Dox, and Dox alone, in MF/SS cells, SPBL, and NPBL. The IC₅₀ of AN-7+Dox exhibited strong selectivity in MF/SS cell lines compared to NPBL (MyLa p=0.02, Hut78p=0.003), whereas the IC₅₀ of SAHA+Dox exhibited selectivity in Hut78 cells (p=0.02) and not in MyLa cells (p=0.5).

TABLE 4
IC₅₀ values of AN-7, SAHA alone or combined with Dox,
and Dox alone in different types of cells

| | | | IC₅₀ combined | | | | IC₅₀ combined | |
|---|---|---|---|---|---|---|---|---|
| | IC₅₀ single | | treatment of AN-7 | | IC₅₀ single | | treatment of | |
| | treatment | | and Dox | | treatment | | SAHA and Dox | |
| Cell | AN-7 | Dox | AN-7 | Dox | SAHA | Dox | SAHA | Dox |
| type | (μM) | (μM) | (μM) | (μM) | (μM) | (μM) | (μM) | (μM) |
| Myla | 81.67 ± 4.4 | 19 ± 1 | 28.3 ± 0.8 | 9.4 ± 0.29 | 2.2 ± 0.1 | 19 ± 1 | 2.1 ± 0.3 | 13.96 ± 2 |
| Hut78 | 59 ± 4.3 | 27 ± 2.6 | 27.3 ± 2.85 | 10.5 ± 1.1 | 0.5 ± 0.0 | 27 ± 2.6 | 0.39 ± 0.03 | 10.2 ± 0.9 |
| Sezary | 128 ± 5.2 | 68.2 ± 10.8 | 66.7 ± 10.8 | 25.8 ± 4.3 | 2.96 ± 0.74 | 68.2 ± 18.1 | 1.8 ± 0.4 | 34.3 ± 4.9 |
| PBL | | | | | | | | |
| Normal | 191 ± 26.1 | 399 ± 24.3 | 73.3 ± 12.2 | 41.21 ± 6.8 | 2.84 ± 0.65 | 399 ± 24.3 | 1.73 ± 0.36 | 19.45 ± 5.1 |
| PBL | | | | | | | | |

Differences in selectivity of the combined treatment between SPBL and NPBL failed to reach statistical significance because of the small size of the patients group. The CI-vs.-viability fraction plots demonstrated a synergistic effect of AN-7+Dox in Hut78 cells (FIG. 5I) as well as in SPBL (FIG. 5J). The dose combination of AN-7+Dox leaving less than 50% of viable MyLa cells was also synergistic (FIG. 5G), as opposed to the antagonistic effect in NPBL (FIG. 5J). The CI-vs.-viability fraction plots demonstrated an antagonistic effect of SAHA+Dox in both MyLa and Hut78 cell lines (FIGS. 5G and 5I). The dose combination of SAHA+Dox leaving less than 50% of viable SPBL had an antagonist-to-additive effect, with similar results in NPBL (FIGS. 5I and 5J, respectively).

Table 5 summarizes the CI values at representative viable fractions in each cell type, derived from the curves of FIGS. 5G-J;

TABLE 5
CI values at representative viable fractions

| | Combination index | | | | Combination index | | | |
|---|---|---|---|---|---|---|---|---|
| | AN-7 + Dox | | | | SAHA + Dox | | | |
| | | | Sezary | Normal | | | Sezary | Normal |
| | Myla | Hut78 | PBL | PBL | Myla | Hut78 | PBL | PBL |
| 0.2 | 0.79 | 0.66 | 0.79 | 6.54 | 1.13 | 1.25 | 1.18 | 1.8 |
| 0.4 | 0.84 | 0.73 | 0.67 | 3.28 | 1.32 | 1.3 | 0.94 | 0.95 |
| 0.6 | 1.15 | 0.79 | 0.66 | 2.1 | 1.84 | 1.35 | 0.82 | 0.61 |
| 0.8 | 2.27 | 0.87 | 0.81 | 1.45 | 3.61 | 1.41 | 0.74 | 0.39 |

### Example 5

### AN-7 Prevents the Repair of Dox-Induced DNA Breaks in CTCL Cell Lines

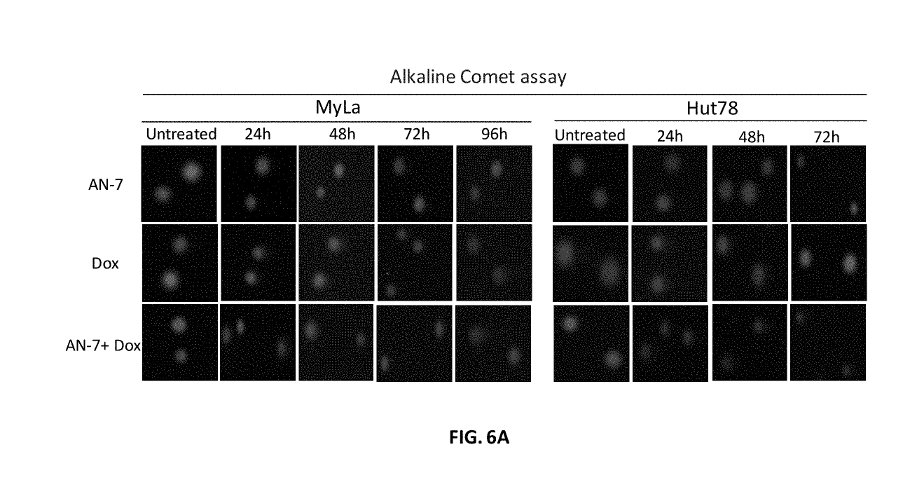

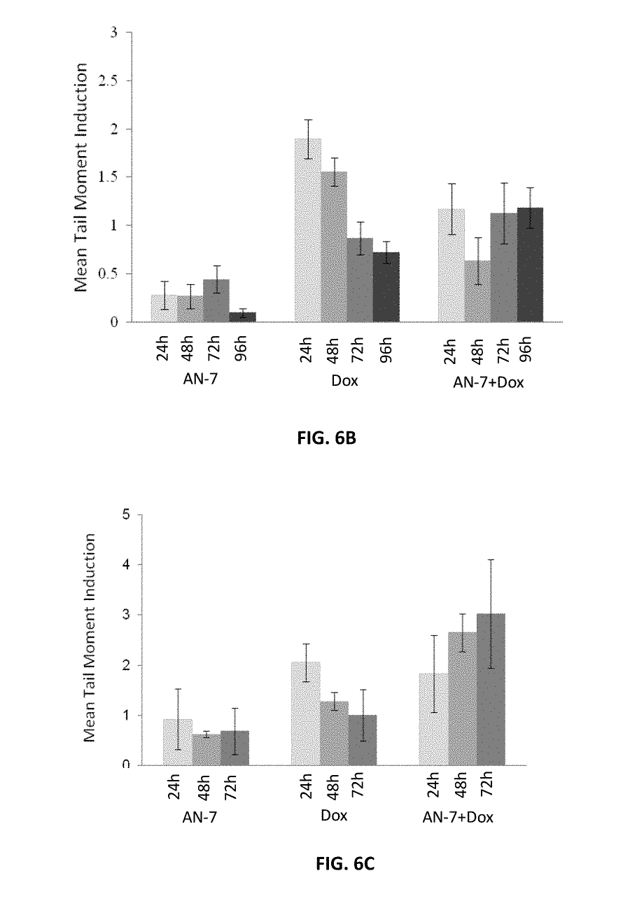

First the effect of AN-7 on the induction and repair of DNA strand breaks induced by Dox was tested via alkaline comet assay. MyLa and Hut78 cells were treated with AN-7 for 2 h, Dox for 1 h, or the combination of AN-7 for 1 h followed with Dox for another 1 h, washed and incubated for interval times of 24 h-96 h. Cells embedded on microscope slides were lysed to form DNA linked to the nuclear matrix followed by single cell electrophoresis. The length of the comet tail relative to head reflects the numbers of DNA breaks. The maximal tail moment length was detected 24 h after treatment with Dox, followed by decrease in the tail moment at 48-96 h, which indicates for the repair of DNA breaks (FIG. 6A-C). The repair of the DNA breaks is nicely presented in MyLa cells treated with Dox as a gradual reduction in the mean tail moment between 48-96 h after damage induction, with damage repair of about 72% after 96 h (FIG. 6A), while 96 h after the combined treatment there was no changes in the mean tail moment indicating for DNA repair inhibition. Also Hut78 cells treated with Dox demonstrated repair of about 50% of the break after 72 h, while cells treated with AN-7+Dox exhibited no repair and even accumulation of DNA breaks (FIG. 6B). The difference in the mean tail moment of MyLa cells with Dox to that of Dox with AN-7 at 48-96 h was not significant, but in Hut78 cells was significant at 48-72 h (p=0.02). These results indicate that in CTCL cell lines, AN-7 abolishes the repair of DNA breaks induced by Dox.

### Example 6

### AN-7 Mediates Prolong Phosphorylation of DDR Markers in CTCL Cell Lines

Since DSBs are the most harmful DNA lesion, the effect of AN-7 on the induction and repair of DSBs was examined by following the kinetic appearance and clearance of phosphorylated KAP1 and γH2AX (common DSB markers) in western blot.

To this end, MyLa cell were treated with 1 mM AN-7 for 2 hours, 1 μM Dox for 1 hour, or a combination of AN-7 for 1 hour followed with Dox for another 1 hour. Hut78 cells were treated with 0.5 mM AN-7 for 2 h, 0.5 μM Dox for 1 h, or a combination of AN-7 for 1 hour followed with Dox for another 1 hour. Next, cells were fractionated and fraction lysate were subjected to western blot of phosphorylated KAP1 (p-KAP1), γH2AX and actin.

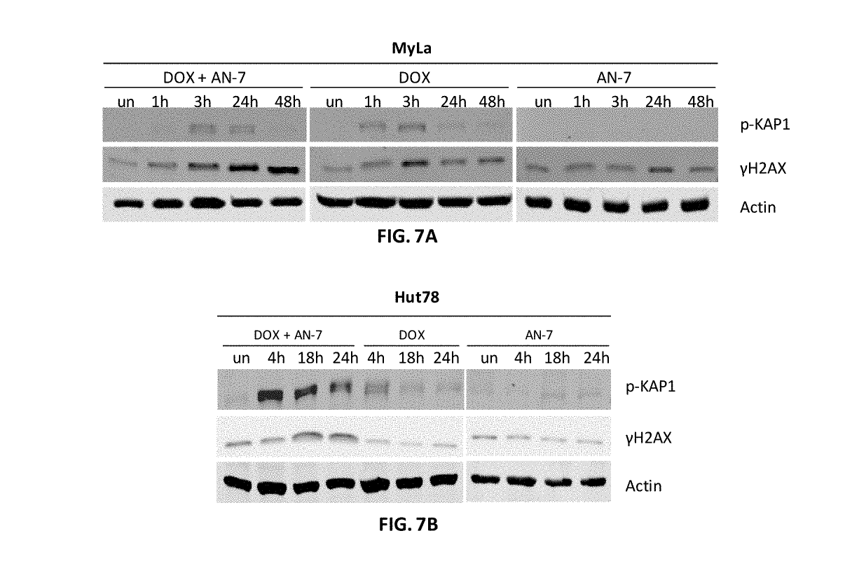

The induction of p-KAP1 in MyLa cells treated with Dox was detected at 1-3 hours and declined after 24 hours (FIG. 7A). While, in the combined treatment, the induction appeared also at 3 h, but lasted at 24 h, and cleared only after 48 h (FIG. 7A). The pic induction of γH2AX in MyLa cells was also detected at 3 h following treatment with Dox alone and combined with AN-7, while the clearance of γH2AX observed at 24 h post Dox treatment, was completely abolished in the combined treatment and persisted even at 48 h (FIG. 7A). In Hut78 cells the combined treatment leaded to higher induction of p-KAP1 and γH2AX at 4h compared to Dox alone, and persisted longer (FIG. 7B). Single treatment of AN-7 did not affect the expression of p-KAP1 and γH2AX in both CTCL cell lines (FIG. 7B). The persistence phoshphorylation of KAP-1 and H2AX induced by Dox due to pre-treatment with AN-7 indicates for the interference of AN-7 in the repair of Dox-induced DSBs.

### Example 7

### AN-7 Facilitates Sustain Dox-Induced γH2AX Nuclear Foci in CTCL Cell Lines

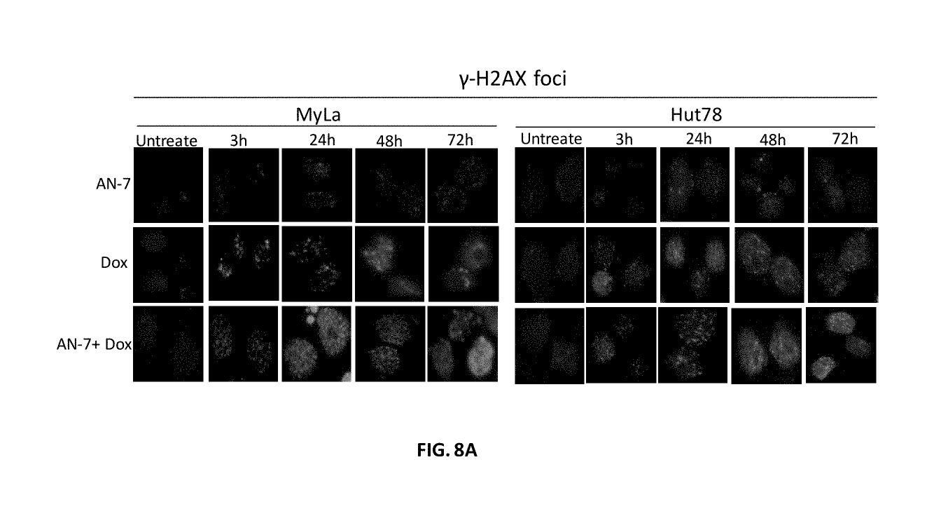

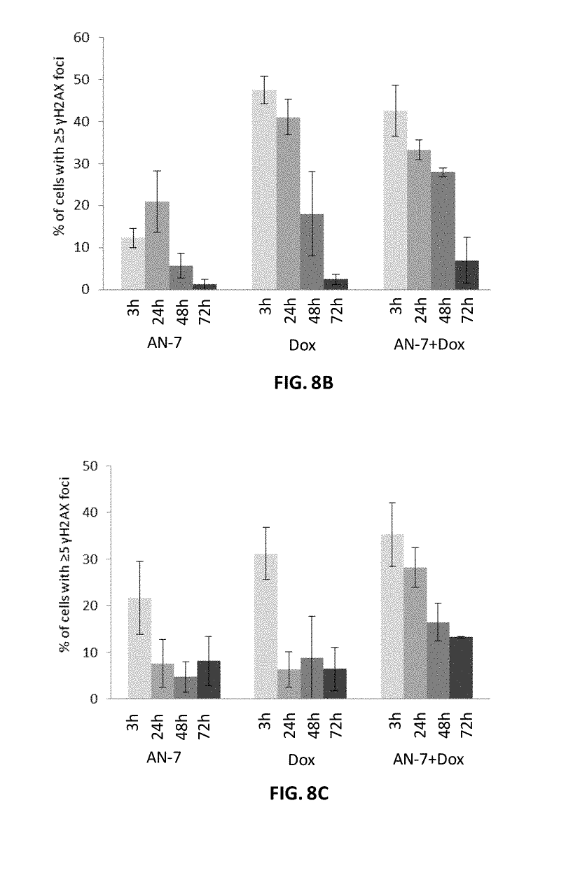

γH2AX nuclear foci are microscopically visible subnuclear foci and then decreased over time of 24-72 h, with attenuated decrease in the combined treatment of Dox+AN-7 (FIGS. 8A and B). The percentage of MyLa cells with residual γH2AX foci (of at least 5 foci/cell) was higher at 48 and 72 h after the combined treatment compared to Dox alone but with no significance (p=0.47) (FIGS. 8A and 8B). While in Hut78 cells the percentage of cells with residual γH2AX foci was significantly higher in the combined treatment compared to Dox at 24-72 h (p=0.0167) (FIGS. 8A and 8C). The percentage of repaired damage in Hut78 cells at 24 h-72 h after combined treatment was lower than in the Dox alone (43.4% compared to 77.9%, p=0.0134). The γH2AX foci results support the western blot findings confirming the role of AN-7 in inhibiting the repair of DSBs induced by Dox, rather than enhancing the formation of DSBs in CTCL cell lines.

### Example 8

### AN-7 Down-Regulates the Expression of DSB Repair Proteins in CTCL Cell Lines

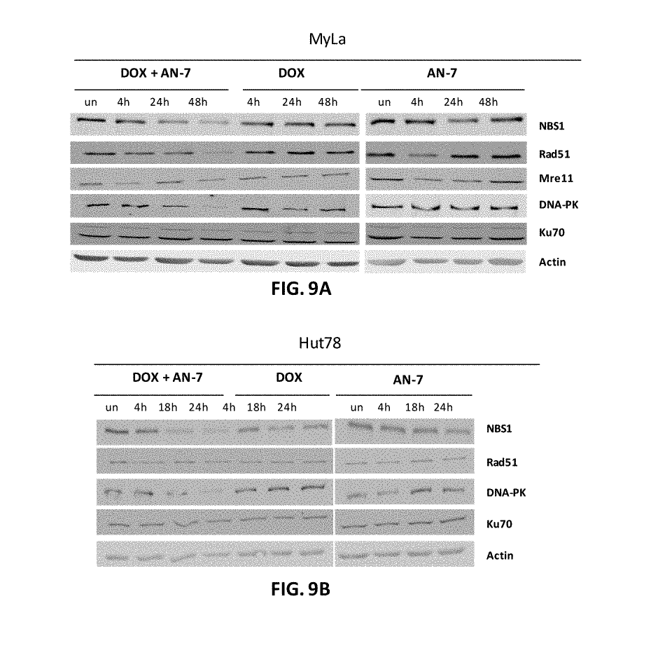

To address whether AN-7 interfere in DSBs repair by regulating the expression of DSBs repair proteins, Immunoblot was applied for those proteins. MyLa and Hut78 cells were treated as previously described and subjected to western blot of DSBs repair proteins. It was found that single treatment with AN-7 or Dox did not affect the expression of DSB repair proteins, while the combination of AN-7 with Dox down-regulate the expression of DSB repair proteins from either the homologous recombination (HR) or the non-homologous end joining (NHEJ) pathway in CTCL cell lines (FIG. 9A-B). NBS1, Mre11 and Rad51, key players of the HR DSB repair, and DNA-PK from the NHEJ were down regulated in MyLa cells 24 and 48 h post treatment with AN-7+Dox, while Ku70 was unaffected (FIG. 9A). Downregulation of NBS1 and DNA-PK were also obtained in Hut78 cells 18 and 24 h post treatment with AN-7 +Dox, while Rad51 was unaffected and the expression of Mre11 was undetectable (FIG. 9B).

### Example 9

### AN-7 Suppresses HR-Direct DSBs Repair

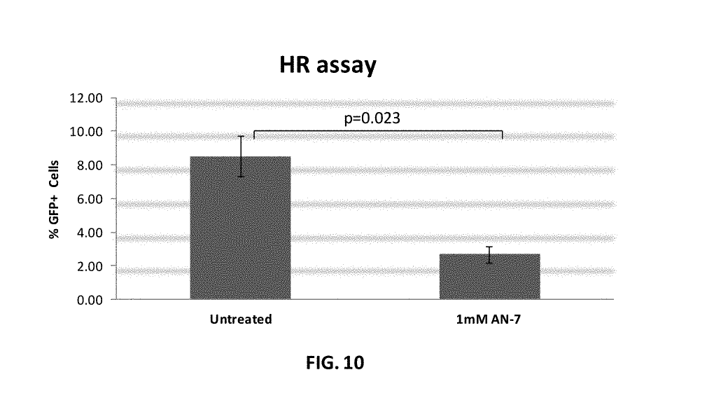

The role of AN-7 in suppressing directly the repair of DSBs, was verified. To this end, one of the machinery for repairing DSBs, the HR repair machinery, was tested. The U2OS-GFP based approach was utilized to score for the effect of AN-7 on the HR efficiency in repairing DSBs induced by I-SceI. The experimental system is based on U2OS cells in which interrupted GFP encoding sequences containing recognition sites of the rare cutter restriction endonuclease I-SceI were incorporated into the cellular genome. The repair of I-SceI-induced DSB via HRR regenerates an active GFP encoding sequence. The U2OS-GFP cells were transfected with I-SceI plasmid for 24 hours, treated then with AN-7 for another 48 h and then analyzed for GFP expression in FACS analysis. GFP-positive cells were gated, and the percentage of GFP-positive cells was normalized against that of cells transfected with GFP control vector. AN-7 was found to significantly reduce the proportion of cells expressing GFP (p=0.023) which point the reduction in the frequency of HR repair (FIG. 10).

### Example 10

### In Vivo Effect of AN7 Alone or Combined with Doxorubicin

In order to generate a xenograft mouse model of MF, a NOD scid gamma (NSG) female mice aged 12 weeks are subcutaneously injected at the lower limbs with 1×10⁷ HUT78 cells in 100-200 μL of buffered saline. Beige SCID female mice at ages 7-8 weeks are subcutaneously injected at the back axil with 1×10⁷ MyLa cells in 100-200 μL of buffered saline. The mice are raised in the pathogen-free animal facility. Tumors typically appear at the sites of injection within 2-3 weeks.

The effect of AN-7 alone or combined with on the survival, proliferation, and growth of MyLa or HUT78 cells is tested in vivo in the xenograft mouse model of MF. To this end, mice are treated with AN-7 at doses of 20-200 mg/kg body weight dissolved in saline or PBS that can be given orally or intraperitoneally (IP), three times a week for three weeks. Doxorubicin as a single agent is given intraperitoneally (IP) or intravenously (IV) once a week for three weeks at doses of 0.5-8 mg/kg body weight dissolved in saline or PBS. In the combined treatment, AN-7 is given orally, three times a week, for three weeks at doses of 10-100 mg/kg body weight dissolved in saline or PBS and doxorubicin is given intraperitoneally (IP) or intravenously (IV) once a week for three weeks at doses of 2-10 mg/kg.

What is claimed is:

1. A method for treating ameliorating, reducing and/or preventing a cutaneous T-cell lymphoma (CTCL) in a subject in need thereof, said method comprising the step of: administering to the subject a therapeutically effective amount of butyroyloxymethyl diethyl phosphate (AN-7) a derivative or salt thereof, thereby treating, ameliorating, reducing and/or preventing a cutaneous T-cell lymphoma in a subject in need thereof.

2. The method of claim 1, wherein said cutaneous T-cell lymphoma (CTCL) is selected from the group consisting of: Mycosis fungoides and Sézary syndrome.

3. The method of claim 1, further comprising the step of simultaneously, sequentially or separately administering or applying an additional anti-cancer therapy selected from the group consisting of: a radiation therapy and an anti-cancer agent.

4. The method of claim 3, wherein said anti-cancer agent is a topoisomerase inhibitor.

5. The method of claim 4, wherein the topoisomerase inhibitor is selected from the group consisting of: doxorubicin, epirubicin, daunomycin, amscrine, and mitoxantrone.

6. A method for treating ameliorating, reducing and/or preventing a cutaneous T-cell lymphoma (CTCL) in a subject in need thereof, said method comprising the step of: simultaneously, sequentially or separately administering to the subject a therapeutically effective amount of butyroyloxymethyl diethyl phosphate (AN-7), a derivative or salt thereof, and an additional anti-cancer therapy, thereby treating, ameliorating, reducing and/or preventing a cutaneous T-cell lymphoma (CTCL) in a subject in need thereof.

7. The method of claim 6, for increasing a therapeutic potency, efficacy or selectivity of said anti-cancer therapy.

8. The method of claim 6, wherein said additional anti-cancer therapy is selected from the group consisting of: a radiation therapy and an anti-cancer agent.

9. The method of claim 8, wherein said anti-cancer agent is a topoisomerase inhibitor.

10. The method of claim 9, wherein said topoisomerase inhibitor is selected from the group consisting of: doxorubicin, epirubicin, daunomycin, amscrine, and mitoxantrone.

11. A method for inducing cell death, proliferation arrest, or growth arrest of a neoplastic T-cell in a cutaneous T-cell lymphoma, said method comprising the step of contacting said neoplastic T-cell with butyroyloxymethyl diethyl phosphate (AN-7), a derivative or salt thereof, thereby inducing cell death, proliferation arrest, or growth arrest.

12. The method of claim 11, further comprising the step of simultaneously, sequentially, or separately contacting said neoplastic T-cell with an anti-cancer agent.

13. The method of claim 11, wherein said cutaneous T-cell lymphoma (CTCL) is selected from the group consisting of: Mycosis fungoides and Sézary syndrome.

14. The method of claim 12, wherein said anti-cancer agent is a topoisomerase inhibitor.

15. The method of claim 14, wherein said topoisomerase inhibitor is selected from the group consisting of: doxorubicin, epirubicin, daunomycin, amscrine, and mitoxantrone.
